(19) United States
(12) Patent Application Publication
(10) Pub. No.: US 2025/0302650 A1
(43) Pub. Date: Oct. 2, 2025

(54) MODULAR STENTS

(71) Applicant: EDWARDS LIFESCIENCES CORPORATION, Irvine, CA (US)

(72) Inventors: Roei Mayo, Irvine, CA (US); Ben Shitrit, Eilat (IL); Arvin T. Chang, Yorba Linda, CA (US); Anthony Ciro Vrba, San Clemente, CA (US); Daniel James Murray, Orange, CA (US); Leonardo Paim Nicolau Da Costa, Tampa, FL (US); Scott Louis Pool, Laguna Hills, CA (US); Amanda Tjipta Sugijoto, Tustin, CA (US)

(73) Assignee: EDWARDS LIFESCIENCES CORPORATION, Irvine, CA (US)

(21) Appl. No.: 19/236,819

(22) Filed: Jun. 12, 2025

Related U.S. Application Data
(63) Continuation of application No. PCT/US2024/010737, filed on Jan. 8, 2024.
(60) Provisional application No. 63/481,135, filed on Jan. 23, 2023.

(51) Int. Cl. A61F 2/966; A61F 2/06; A61F 2/82

(52) U.S. Cl. CPC A61F 2/966; A61F 2002/068; A61F 2002/826; A61F 2210/0014; A61F 2220/0025; A61F 2230/0008; A61F 2230/001; A61F 2250/006

(57) ABSTRACT

A method of managing blood flow involves advancing a delivery system to a blood vessel segment and deploying a first stent from the first delivery system at a first position in the blood vessel segment, the first stent having a non-circular cross-sectional shape defining a major-axis diameter and a minor-axis diameter that is less than the major-axis diameter. A second stent is deployed from the delivery system at a second position spaced from the first position by a first axial gap, the second stent having the non-circular cross-sectional shape and being physically coupled to the first stent by first and second coupling arms positioned on opposite major-axis circumferential portions of the first and second stents, respectively. Systolic pressure is reduced through circularization of the first and second stents and diastolic pressure is increased through shape-memory return of the first and second stents to non-circular shapes.

### RELATED APPLICATIONS

This application is a continuation of International Patent Application No. PCT/US24/10737, filed Jan. 8, 2024, which claims the benefit of U.S. Provisional Patent Application No. 63/481,135, filed on Jan. 23, 2023, the complete disclosures of which are hereby incorporated by reference in their entireties.

### BACKGROUND

The present disclosure generally relates to the field of medical implant devices, including stent implant devices. Stent implant devices can be designed for intravascular deployment. The length, shape, and/or configuration of a stent implant device can impact the suitability of such devices for implantation in particular vascular anatomy of a patient.

### SUMMARY

Described herein are devices, methods, and systems relating to stent devices/assemblies comprising two or more stents, or stent segments, configured to be implanted in axially-offset relative positions, as an alternative to a single, relatively long stent having similar overall length. Such stent segments can advantageously have a non-circular biased axial cross-sectional shape, such that the stent segments can naturally, due to spring biasing thereof, cyclically alternate between more-circular (e.g., circular) and less-circular (e.g., oval) shapes/configurations between relatively high (e.g., systole) and low (e.g., diastole) pressure states, thereby evening-out flood flow in the target blood vessel by cyclically reshaping the target blood vessel. In some implementations, adjacent stent segments are coupled by connecting arms/struts, which may be positioned on, or in the area of, major-axis ends/walls of non-circular stent segments. Such connecting arms/struts can serve to align adjacent stent segments and/or deform/shape the gap portions of the target blood vessel between the connected stent segments. Stent-segment-connecting arms/struts can be configured to bend in at least one plane/dimension, whereas bending/deflection of the arms/struts may be limited/restricted in at least one plane/dimension relative to the bending plane/dimension.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Any of the example methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIGS. 1A and 1B show front and side views, respectively, of a human, including views of certain internal anatomy.

FIGS. 2A and 2B show side and axial cross-sectional views, respectively, of a compliant blood vessel experiencing compliant expansion and contraction over a cardiac cycle.

FIG. 3A shows an example stiff aorta.

FIGS. 3B and 3C show side and axial cross-sectional views, respectively, of a stiff blood vessel experiencing compromised expansion and contraction over a cardiac cycle.

FIG. 4 shows cutaway view of a stent disposed in a blood vessel in accordance with one or more examples.

FIGS. 5-1 and 5-2 show a blood vessel in circular and non-circular shapes, respectively.

FIGS. 6A-6D show perspective, side, and axial views, respectively, of a non-circular stent segment in accordance with one or more examples.

FIG. 7 shows an axial view of a non-circular stent segment deployed within a blood vessel in accordance with one or more examples.

FIG. 8 shows a stent spanning a curved blood vessel segment in accordance with one or more examples.

FIG. 9A shows a cutaway view of modular, non-circular stent segments placed in series within a blood vessel segment in accordance with one or more examples.

FIG. 9B shows a cutaway view of modular, non-circular stent segments placed in series within a blood vessel segment in accordance with one or more examples.

FIG. 10A shows a view of a minor-axis side of a modular stent including non-circular stent segments coupled by link/connector features in accordance with one or more examples.

FIG. 10B shows a view of a major-axis side of the modular stent of FIG. 10A in accordance with one or more examples.

FIG. 10C shows an axial view between the stent segments of the modular stent of FIGS. 10A and 10B.

FIG. 11A shows a view of a minor-axis side of a modular stent implanted in a blood vessel segment in accordance with one or more examples.

FIG. 11B shows a view of a major-axis side of the modular stent of FIG. 11A implanted in the blood vessel segment in accordance with one or more examples.

FIG. 11C shows an axial view between the stent segments of the modular stent of FIGS. 11A and 11B.

FIGS. 12A and 12B show perspective and axial views, respectively, of a modular stent comprising peanut-shaped stent segments in accordance with one or more examples.

FIG. 13 shows a modular stent including connecting arms implanted in a curved blood vessel segment in accordance with one or more examples.

FIG. 14 shows a cutaway view of a delivery system for delivering a modular stent in accordance with one or more examples.

FIG. 15 shows a modular stent including a covering that connects stent segments in accordance with one or more examples.

FIG. 16 is a flow diagram illustrating a process for managing flow in a target blood vessel using a modular stent in accordance with one or more examples.

FIGS. 17-1, 17-2, 17-3, 17-4, and 17-5 illustrate a flow diagram for a process for deploying a modular stent implant including a sleeve component in accordance with one or more examples.

FIGS. 18-1, 18-2, 18-3, 18-4, and 18-5 provide images of aspects of the modular stent, delivery system components, and anatomy relating to operations of the process of FIGS. 17-1, 17-2, 17-3, 17-4, and 17-5 according to one or more examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Vascular Anatomy and Compliance

Certain examples are disclosed herein in the context of vascular implant devices, and in particular, stent-shaping implant devices implanted in the aorta. However, although certain principles disclosed herein may be particularly applicable to the anatomy of the aorta, it should be understood that modular stent implant devices in accordance with the present disclosure may be implanted in, or configured for implantation in, any suitable or desirable blood vessels or other anatomy, such as the inferior vena cava.

The anatomy of the heart and vascular system is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves.

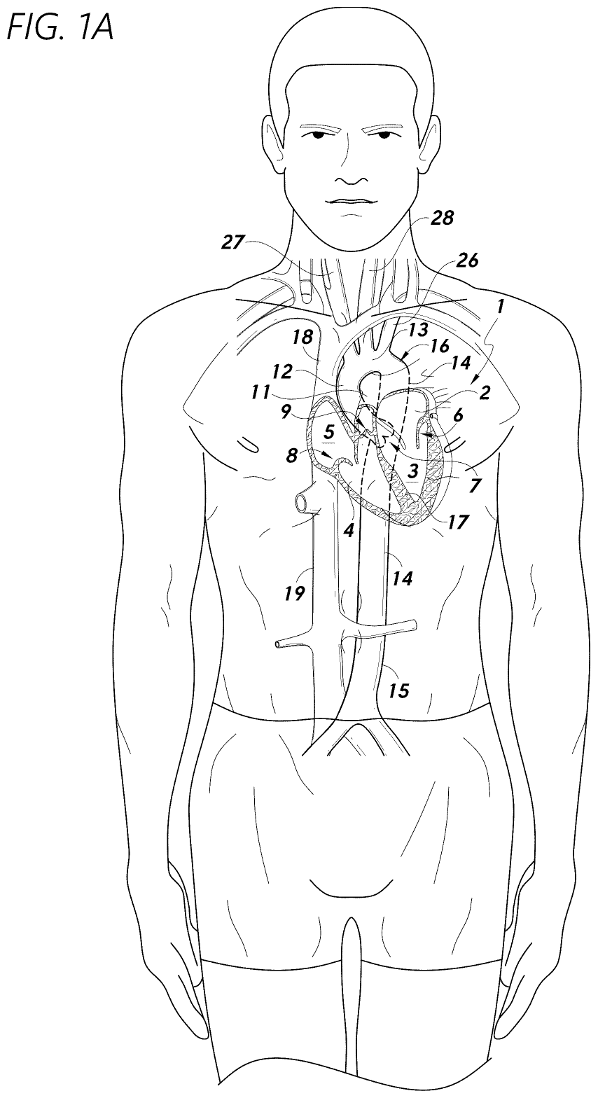

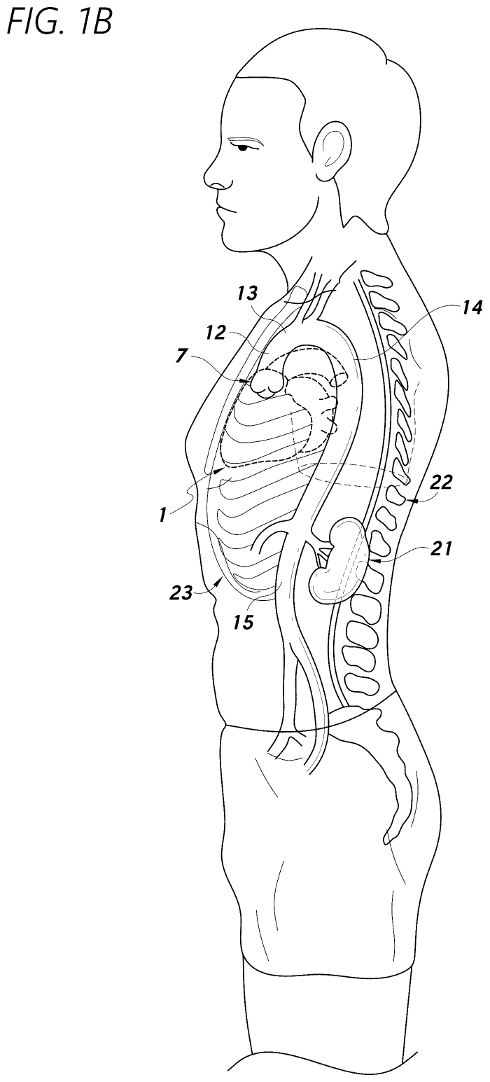

FIG. 1A and FIG. 1B show front and side views, respectively, of a human, including views of certain internal anatomy, such as a heart 1 and associated vasculature having various features relevant to one or more examples of the present inventive disclosure. The heart 1 includes four chambers, namely the left atrium 2, the left ventricle 3, the right ventricle 4, and the right atrium 5, which are separated by the atrioventricular heart valves. The various heart valves may be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles may be prompted by signals generated by the electrical system of the heart.

In terms of blood flow, blood generally flows from the right ventricle 4 into the pulmonary artery via the pulmonary valve 9, which separates the right ventricle 4 from the pulmonary artery 11 and is configured to open during systole so that blood may be pumped toward the lungs and close during diastole to prevent blood from leaking back into the heart from the pulmonary artery 11. The pulmonary artery 11 carries deoxygenated blood from the right side of the heart to the lungs. The pulmonary artery 11 includes a pulmonary trunk and left and right pulmonary arteries that branch off of the pulmonary trunk, as shown.

The tricuspid valve 8 separates the right atrium 5 from the right ventricle 4. The tricuspid valve 8 generally has three cusps/leaflets and may generally close during ventricular contraction (i.e., systole) and open during ventricular expansion/dilation (i.e., diastole). The mitral valve 6 generally has two cusps/leaflets and separates the left atrium 2 from the left ventricle 3. The mitral valve 6 is configured to open during diastole so that blood in the left atrium 2 can flow into the left ventricle 3, and, when functioning properly, closes during systole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

The atrioventricular (mitral and tricuspid) heart valves generally are coupled to a collection of chordae tendineae and papillary muscles (not shown for visual clarity) for securing the leaflets of the respective valves to promote and/or facilitate proper coaptation of the valve leaflets and prevent prolapse thereof. The papillary muscles, for example, may generally comprise finger-like projections from the ventricle wall. The valve leaflets are connected to the papillary muscles by the chordae tendineae. A wall of muscle 17, referred to as the septum, separates the left 2 and right 5 atria and the left 3 and right 4 ventricles.

The vasculature of the human body, which may be referred to as the circulatory system, cardiovascular system, or vascular system, contains a complex network of blood vessels with various structures and functions and includes various veins (venous system) and arteries (arterial system). Generally, arteries, such as the aorta 16, carry blood away from the heart, whereas veins, such as the inferior 19 and superior 18 venae cavae, carry blood back to the heart.

The aorta 16, which is of particular significance with respect to certain inventive examples disclosed herein, is a compliant arterial blood vessel that buffers and conducts pulsatile left ventricular output and contributes the largest component of total compliance of the arterial tree. The aorta 16 may be considered to include various portions/segments, including the ascending aorta 12, which begins at the opening of the aortic valve 7 in the left ventricle of the heart. The ascending aorta 12 and pulmonary trunk 11 generally twist around each other, causing the aorta 12 to start out posterior to the pulmonary trunk 11, but end by twisting to its right and anterior side. Among the various segments of the aorta 16, the ascending aorta 12 is relatively more frequently affected by aneurysms and dissections, often requiring open heart surgery to be repaired. The transition from the ascending aorta 12 to the aortic arch 13 is typically at the pericardial reflection on the aorta. At the root of the ascending aorta 12, the lumen has three small pockets between the cusps of the aortic valve and the wall of the aorta, which are called the aortic sinuses, or the sinuses of Valsalva. The left aortic sinus contains the origin of the left coronary artery and the right aortic sinus gives rise to the right coronary artery. Together, these two arteries supply the heart.

As mentioned above, the aorta 16 is coupled to the heart 1 via the aortic valve 7, which leads into the ascending aorta 12 and gives rise to the innominate artery 27, the left common carotid artery 28, and the left subclavian artery 26 along the aortic arch 13 before continuing as the descending thoracic aorta 14 and further the abdominal aorta 15. References herein to the aorta may be understood to refer to the ascending aorta 12 (also referred to as the “ascending thoracic aorta”), aortic arch 13, descending or thoracic aorta 14 (also referred to as the “descending thoracic aorta”), abdominal aorta 15, or other arterial blood vessel or portion thereof.

Arteries, such as the aorta 16, may utilize blood vessel compliance (e.g., arterial compliance) to store and release energy through the stretching of blood vessel walls. The term “compliance” is used herein according to its broad and ordinary meaning, and may refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

As with other blood vessels of the body, the aorta may have certain curvature/tortuosity, as shown in the images of FIGS. 1A and 1B. For example, while the aorta may run generally vertically from the junction of the aortic arch 13 and the descending aorta 14 down into the abdominal space, in one or more areas/segments of the aorta 16, the blood vessel may curve/angle laterally (e.g., left/right with respect to the orientation of FIG. 1A) and/or transversely (e.g., left/right with respect to the orientation of FIG. 1B), producing an at least partially tortuous path of the blood vessel. Such curvature/tortuosity of the aorta 16 may serve to conform to and/or accommodate various organs and/or other anatomy. For example, the aorta 16 may curve around the heart 1 and jut forward to some degree below the heart to accommodate the spine 22, ribs 23, kidneys 21, and/or other anatomy. The aorta 16 may further traverse the abdomen in a manner as to service the various organs of the body that draw blood from the arterial system.

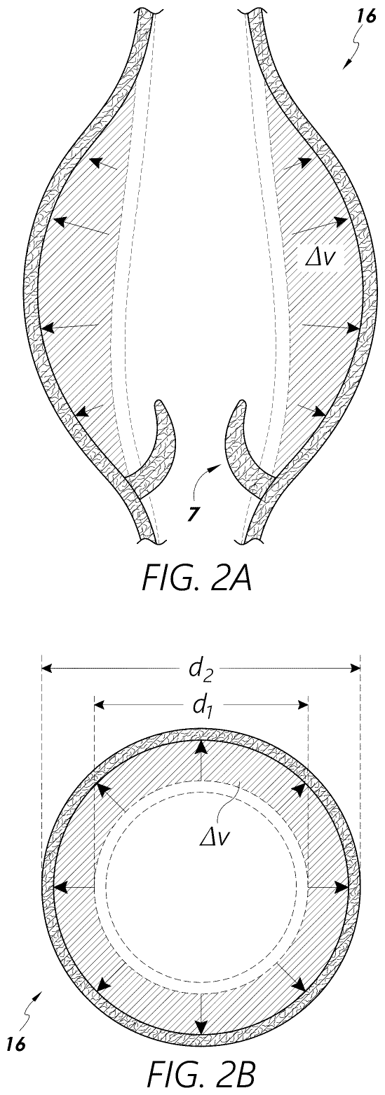

FIGS. 2A and 2B show side and axial cross-sectional views, respectively, of a compliant blood vessel, such as the aorta 16 described above, experiencing compliant expansion and contraction over a cardiac cycle. As referenced above, the systolic phase of the cardiac cycle is associated with the pumping phase of the left ventricle, while the diastolic phase of the cardiac cycle is associated with the resting or filling phase of the left ventricle. As shown in FIGS. 2A and 2B, with proper arterial compliance, an increase in volume Δv will generally occur in an artery when the pressure in the artery is increased from diastole to systole. As blood is pumped into the aorta 16 through the aortic valve 7, the pressure in the aorta increases and the diameter of at least a portion thereof expands. A first portion of the blood entering the aorta 16 during systole may pass through the artery during the systolic phase, while a second portion (e.g., approximately half of the total blood volume) may be stored in the expanded volume Δv caused by compliant stretching of the blood vessel 16 from a non-expanded diameter d₁ to an expanded diameter d₂, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance may have a significant effect on perfusion and/or blood pressure in some patients. For example, arteries with relatively higher compliance may be conditioned to more easily deform than lower-compliance arteries under the same pressure conditions. Compliance (C) may be calculated using the following equation, where Δv is the change in volume (e.g., in mL) of the blood vessel, and Δp is the pulse pressure from systole to diastole (e.g., in mmHg):

$$C = \frac{Δ v}{Δ p} \qquad ( 1 )$$

In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood may flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles and peripheral edema, for example, which may be caused by structural and/or functional cardiac abnormality. Such conditions can result in reduced cardiac output and/or elevated intra-cardiac pressures at rest or during stress.

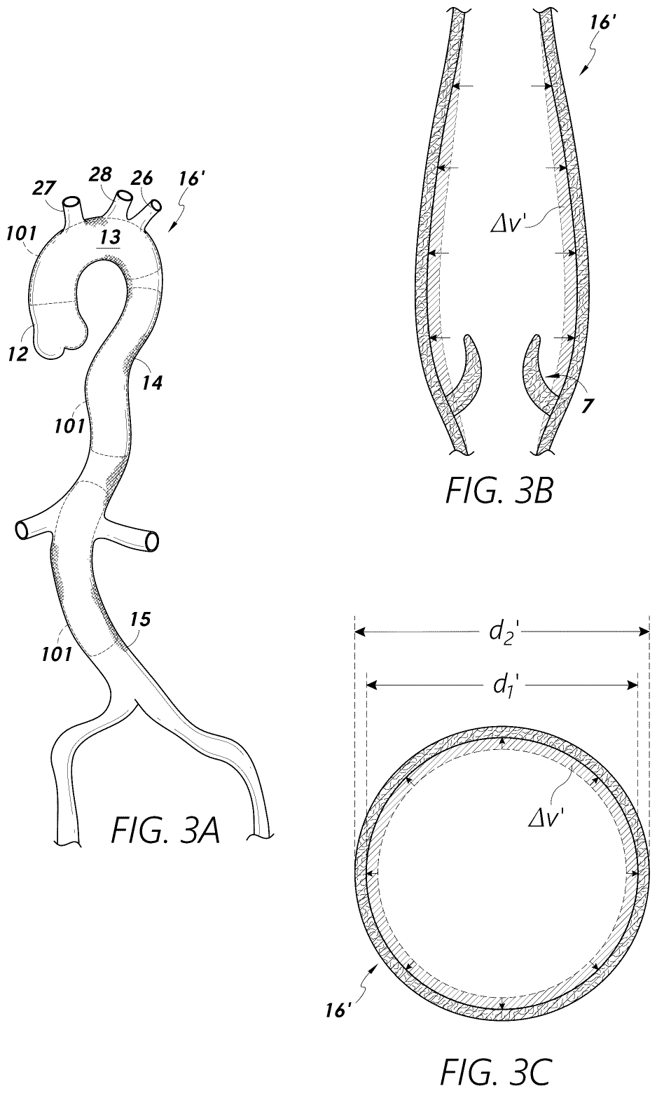

FIG. 3A shows an example stiff aorta 16′. FIGS. 3B and 3C show side and axial cross-sectional views, respectively, of the stiff aorta 16′ of FIG. 3A experiencing compromised expansion and contraction over a cardiac cycle.

As shown in FIG. 3A, the aorta tends to change in shape as a function of age, resulting in a higher degree of curvature and/or tortuosity over time. As the vasculature of a subject becomes less elastic, arterial blood pressure (e.g., left-ventricular afterload) becomes more pulsatile, which can have a deleterious effect. For example, undesirably pulsatile arterial blood flow, such as the thickening of the left ventricle muscle and/or diastolic heart failure. Stiffness in the aorta and/or other blood vessel(s) can occur due to an increase in collagen content and/or a corresponding decrease in elastin.

With the walls of the blood vessel 16′ being resistant to stretching due to the stiffness thereof, the expansion of the blood vessel diameter from the non-expanded diameter d₁′ to the expanded diameter d₂′ may be limited/reduced compared to the expansion of diameter of a healthy blood vessel. Although FIGS. 3B and 3C show a small amount of expansion and volume change Δv′ experienced by the blood vessel 16′, in some cases, a blood vessel may be sufficiently stiff that substantially no vessel expansion takes place in systole.

Generally, the majority of aortic compliance is provided in the ascending aorta 12 with respect to healthy anatomy. Furthermore, calcification frequently occurs in the area of the ascending aorta 12, near the aortic arch 13 and the great vessels emanating therefrom. Such anatomical areas can experience relatively higher stresses due to the geometry, elasticity, and flow dynamics associated therewith. Therefore, implantation/deployment of compliance-enhancing modular stent devices of the present disclosure can advantageously be in the ascending aorta 12 in some cases. While relatively less calcification tends to occur in the descending 14 and abdominal 15 aorta, implant devices of the present disclosure can advantageously be implanted/deployed in such areas as well for the purpose of increasing compliance in the aortic system. Examples of the present disclosure provide modular stent implants comprising one or more non-circular stent segments configured to enhance compliance characteristics of a target blood vessel. For example, FIG. 3A shows example positions of modular stent implants 101 in various potential areas of an aorta 16′.

### Blood-Vessel-Reshaping Stent Implants

The present disclosure relates to systems, devices, and methods for adding-back and/or increasing compliance in the aorta or other arterial (or venous) blood vessel(s) to provide improved perfusion of the heart muscle and/or other organ(s) of the body. Examples of the present disclosure can include a plurality of non-circular stent segments configured to be positioned within a target blood vessel in axially-offset positions so as to reshape portion(s) of the blood vessel to a non-circular shape during the low-pressure phase of the cardiac cycle. Reshaping the blood vessel segment to a non-circular cross-sectional shape can serve to force blood through the blood vessel segment by pushing the blood through the vessel as the vessel volume reduces in connection with stent contraction induced by cyclical drops in blood pressure.

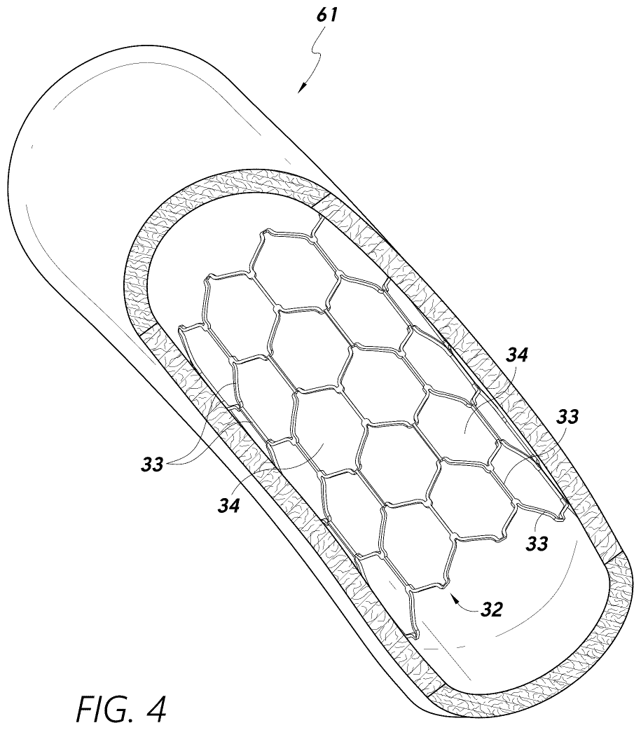

FIG. 4 shows cutaway view of a stent 32 disposed in a blood vessel 61 in accordance with one or more examples. Stents of the present disclosure, including stent segments of modular stent implant devices, can comprise metal (e.g., shape-memory metal, nitinol) or plastic tubes configured to be inserted into the lumen of an anatomic vessel, such as the aorta. Such stents can serve various functionalities, including vessel reshaping to improve and/or even-out blood flow. In some implementations, stent devices are used to hold the vascular passageway open to facilitate blood flow therethrough. Stents and stents segments disclosed herein advantageously may comprise flexible material, such as nitinol or other shape-memory material. The term “stent” is used herein in accordance with its broad and ordinary meaning and may refer to any device configured to be implanted in a lumen of a blood vessel, the device having a tubular form forming a lumen through which blood can flow.

The example stent segment 32 of FIG. 4 may have a frame composed of struts 33 that form open cells 34. Although a particular frame configuration and/or strut pattern is shown in FIG. 4, it should be understood that stent segments disclosed herein may have any suitable or desirable stent frame configuration/pattern configured to be radially compressible and/or expandable for delivery and/or deployment thereof. The stent 32 is shown in the inner diameter of the lumen of a target blood vessel 61, such as the aorta or other arterial or venous blood vessel.

Modular stents of the present disclosure that include stent segments having a biased non-circular shape (e.g., oval- and/or peanut-shaped) can advantageously be configured to reshape a target blood vessel segment to generate a differential cross-sectional area/volume of the target blood vessel (e.g., aorta) between high- and low-pressure phases of the cardiac cycle to facilitate perfusion. As described above, relatively non-compliant blood vessels generally may not be able to stretch to thereby lengthen the perimeter of the blood vessel in response to increased pressure conditions. Such inability to stretch can prevent compliant expansion of the blood vessel.

As described above, desirable diastolic flow in arterial blood vessels is enabled by the decrease in cross-sectional area/volume of the blood vessels when transitioning from higher-pressure conditions (e.g., systole) to lower-pressure conditions (e.g., diastole). Where the relevant blood vessel has become stiff and non-compliant, stretching/expanding and subsequent contraction/shrinking of the blood vessel to cause the desired change in area/volume of the blood vessel may be limited due to the perimeter/wall of the blood vessel being resistant to stretching. Examples of the present disclosure provide implants that cause a change in cross-sectional area/volume of a target blood vessel without requiring stretching in the blood vessel wall. Rather, such cyclical change in blood vessel area/volume can be achieved through manipulation of the shape (e.g., cross-sectional shape) of the target blood vessel, wherein a transition between blood vessel shapes occurring in response to changing pressure conditions can reduce and increase the area/volume of the blood vessel in a cyclical manner to promote more even flow of blood through the blood vessel throughout the cardiac cycle.

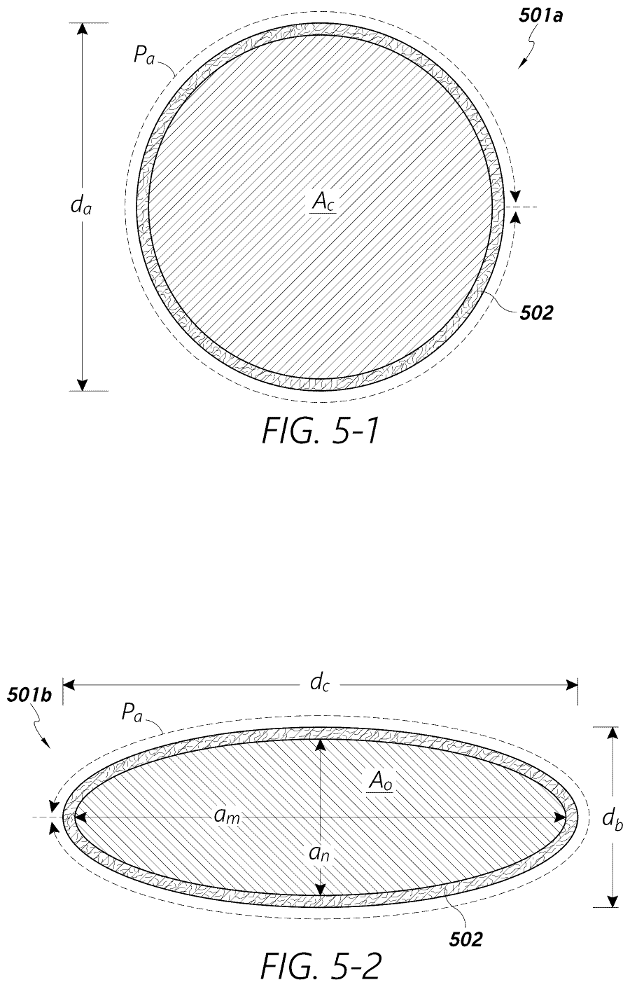

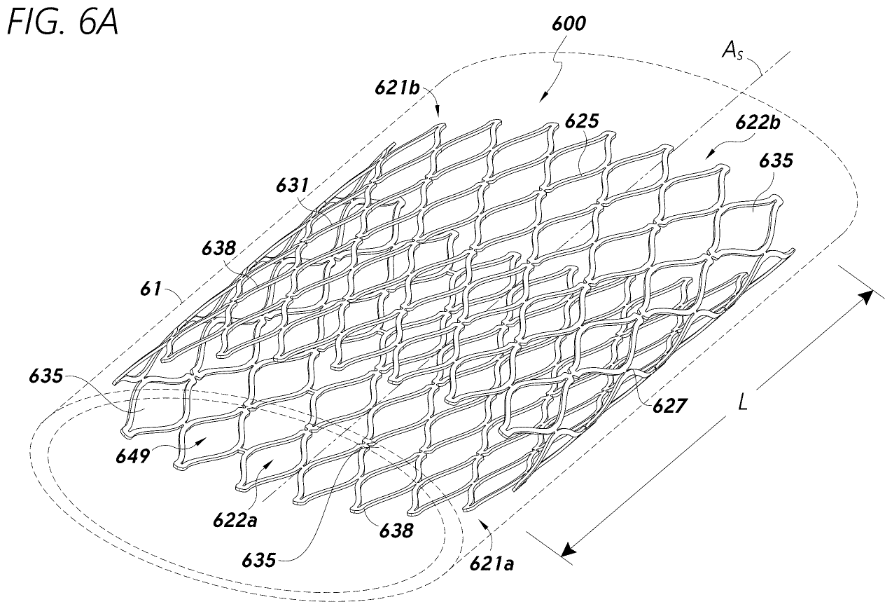

With respect to a blood vessel having a relatively fixed perimeter, wherein the blood vessel wall does not expand sufficiently due to stiffness and/or other factors of non-compliance, generally, the greatest area/volume of the blood vessel may be present/achieved when the blood vessel wall forms a circular cross-sectional shape, which may maximize the cross-sectional area and volume of the blood vessel. FIG. 5-1 shows an example blood vessel 501 (identified as blood vessel 501a in FIG. 5-1) having a generally circular cross-sectional shape, such that the area A_{c} thereof is maximized for the given blood vessel wall perimeter/circumference Pₐ. In the circular configuration, the diameter dₐ is substantially constant at every angle about the axis of the vessel.

Diverging from a circular cross-sectional shape can produce a cross-sectional area/volume for a blood vessel that is less than the maximum area A_{c} shown in FIG. 5-1. For example, FIG. 5-2 shows the blood vessel 501 (identified as vessel 501b in FIG. 5-2) having a shape that resembles an oval/ellipse, which produces a cross-sectional area Aₒ that is less than the area A_{c} with the same blood vessel wall/perimeter length Pₐ. The oval shape of the vessel 501b may have a major axis aₘ having a dimension d_{c} that is greater than a dimension d_{b} of the minor axis aₙ thereof.

With further reference to FIGS. 5-1 and 5-2, due to the area Aₒ of the oval vessel of FIG. 5-1 being less than the area A_{c} of the circular configuration shown in FIG. 5-1, transitioning from the circular shape 501a to the non-circular shape 501b can provide a reduction in area/volume of the blood vessel 501, and therefore solutions that cause transitions between circular and non-circular blood vessel shapes between cardiac phases can provide compliance characteristics without the need for elasticity in the blood vessel wall tissue. For example, where a mechanism is implemented to cause a blood vessel to transition between circular and non-circular shapes in response to changing pressure conditions, such manipulation of the blood vessel shape can introduce volumetric change in the blood vessel in response to the typical changes in pressure experienced during the cardiac cycle, thereby increasing cardiac efficiency and reducing pulsatile load.

In view of the foregoing, examples of the present disclosure provide stent implant devices and associated processes configured to transition the shape/area of a blood vessel from circular/more-circular to non-circular/less-circular shapes, and/or vice versa, to enhance compliance with respect to the area of the implant reshaping. Such stent implant devices/processes may effect vessel reshaping through dynamic reshaping of the structural shape of the stent in a way that produces a change in shape of the blood vessel in which it is implanted to produce a change in blood vessel area/volume between the systolic and diastolic phases of the cardiac cycle.

Examples of the present disclosure provide for modular stent-type implants including separate stent segments that are biased to a non-circular cross-sectional area, such that, in a relaxed/non-pressurized state, a first diameter of the stent segment has a greater dimension along a major axis compared to a second diameter of the stent segment along a minor axis, wherein such stent segments are configured to transition to a more-circular shape when pressure within the blood vessel overcomes the non-circular bias of the stent and causes the stent walls to be pushed and/or pulled to the more-circular configuration. The ability of stent implant devices of the present disclosure to reshape the target blood vessel in the manner described above to produce the desired oval cross-section of the blood vessel can be achievable due to stiff/non-compliant blood vessels, which may be unable to stretch to a substantial degree, still retaining the ability to bend to a sufficient degree to allow for such shaping of the blood vessel. That is, the bending stiffness of a relatively non-compliant blood vessel may be less than the stretching stiffness thereof. Therefore, examples of the present disclosure achieve compliance through bending energy with respect to the blood vessel wall, as opposed to stretching energy. When stents of the present disclosure are forced to a circular, or relatively more-circular, axial cross-sectional shape, energy may be stored in the shape memory of the walls of the stent, wherein recoil/contraction of the stent towards its biased, oval/non-circular configuration can return/release energy to the blood circulation.

As the stents/segments associated with examples of the present disclosure produce complaint blood vessel volume change by manipulating/reshaping the native blood vessel walls, compliance can be increased in the target blood vessel without requiring blood vessel grafting or resection. Therefore, compared to blood flow solutions involving blood vessel grafting/resection, examples of the present disclosure can provide a compliance-enhancing solution that avoids the risks that may be associated with cutting of the vessel and/or devices grafted in/to such vessels, which may present risk of rupture and blood leakage outside of the circulatory system. Hazards associated with extravascular arterial blood leakage, such as within the abdominal and/or chest cavity, can include the risk of serious injury or death.

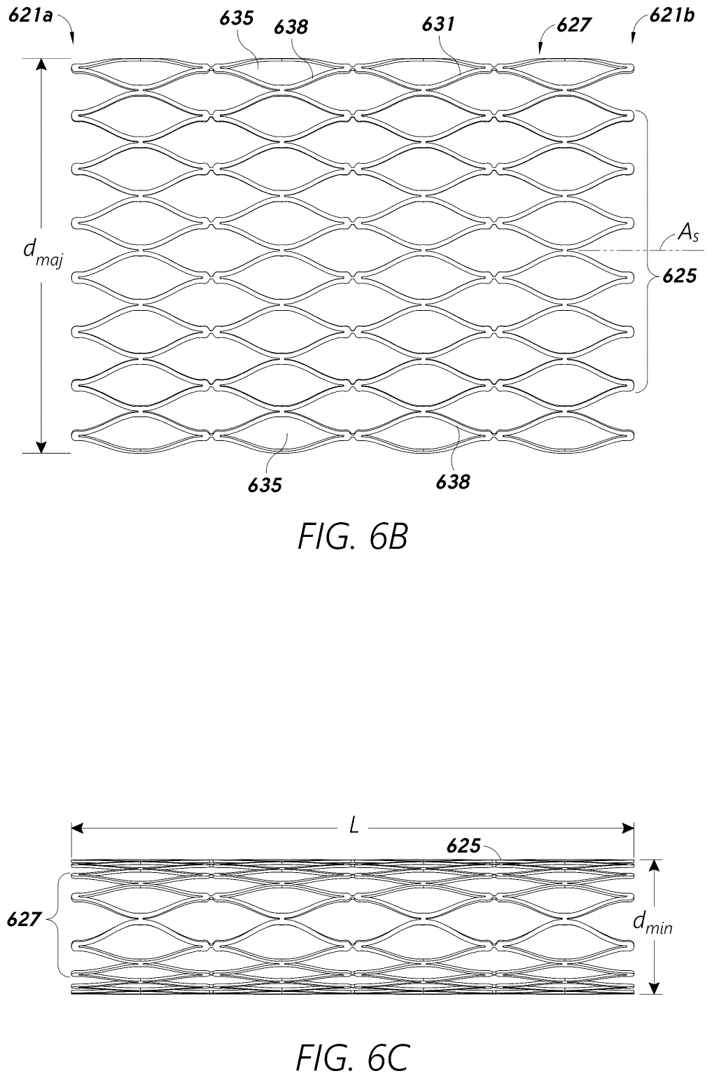

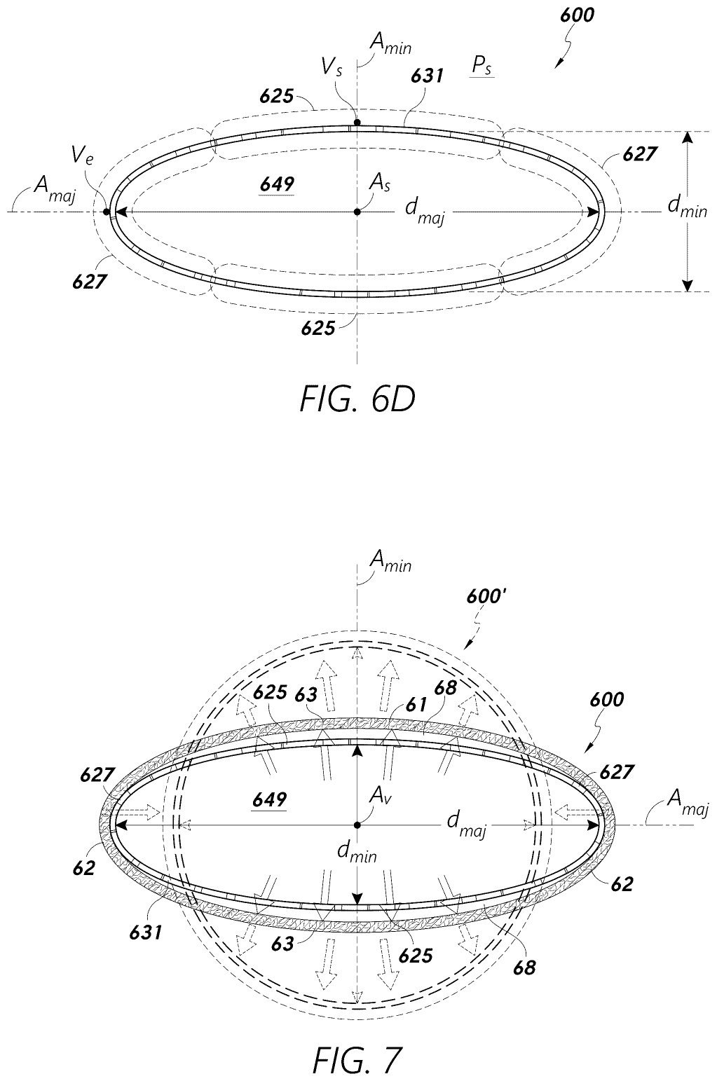

FIGS. 6A-6D show perspective, side, and axial views, respectively, of a non-circular stent segment, which may represent a stent component of any of the modular stent examples disclosed herein. Although a single stent segment 600 is shown, it should be understood that the stent segment 600 may be combined with one or more additional stent segments, which may or may not be coupled by longitudinal connecting arms/struts, to form a modular stent implant as described in detail herein.

The stent segment 600 may be deployable within a blood vessel lumen. However, it should be understood that example modular stent implant devices of the present disclosure and stent segments associated therewith may alternatively or additionally be deployable in a position around an outer surface of a target blood vessel. Although not shown for clarity in the figures of the present disclosure, it should be understood that example stent segments described herein may comprise one or more hooks, barbs, and/or other attachment features/means adapted to facilitate secure attachment of the stent segment to the tissue of the target blood vessel wall.

The stent segment 600 may be formed of a tubular frame 631, which may form a wall around an axial channel 649, thereby defining the channel 649. The stent segment 600 may be an elongate/elongated segment, in that a length L of the stent may be greater than a minor diameter dₘᵢₙ, and/or maximum diameter dₘₐⱼ of the stent segment. As described herein, the frame wall 631 of the stent segment 600 can be considered a single, circumferentially-wrapped wall, or may be considered to comprise multiple walls, or wall segments. For example, with respect to oval stents and other non-circular stents, as illustrated in FIGS. 6A-6D, such stents/segments may be considered to comprise sidewall segments 625 that run along relatively long sides of the stent that are aligned generally with the orientation of the major axis/dimension Aₘₐⱼ of the stent segment, as well as end wall segments 627, which may connect the side walls 625 on major-axis ends/portions of the stent 600. The end walls 627 may be outwardly-curved/concave with respect to an axis Aₛ of the stent 600. The sidewalls 625 may bow/deflect outward, either in a resting, unpressurized state, or in conditions of hoop/wall stress on the frame 631. For example, the sidewalls 625 may bow outward such that the sidewalls 625 are concave from the perspective of the axis Aₛ of the stent segment 600 and convex from the perspective of the exterior of the stent 600.

Certain stent shapes are described herein, including non-circular-, oval-, peanut-, and other-shaped stents. It should be understood that such description of stent shapes refers to a shape of an axial cross-section of a stent segment, as depicted in the view of FIG. 6D. Although oval- and peanut-shaped stent segments are described, it should be understood that the principles of the present disclosure may relate to stents having any non-circular shape in at least some configurations thereof (e.g., in a relaxed/biased configuration), and modular stent implant devices of the present disclosure may comprise stent segments having any combination of circular and/or non-circular shapes. Descriptions of stents in a relaxed or biased configuration should be understood to relate to a configuration that a stent naturally assumes in the absence of tension on the stent wall(s) from external forces (e.g., ambient fluid pressure, physical contact forces, etc.). For example, the biased/relaxed shape of the stent may be due to shape-memory of the stent and/or frame thereof.

The terms “shape memory,” “shape memory effect,” “shape memory characteristic,” and the like are used herein according to their broad and ordinary meanings, and can refer to, for example, any tendency of a material, once deformed, remodeled, adjusted, or otherwise manipulated or configured from an original and/or set/biased shaped thereof, to return to the original/set/biased shape, form, or structure when a deforming force is removed or reduced. For example, in some contexts, shape memory or the like can relate or refer to the ability of a material/element to deform at a temperature when an external force is applied, maintain the deformed shaped when the external force is removed, and return to the undeformed shape when the element is heated above a particular temperature. Further, the terms recited above can connote, indicate, and/or refer to superelasticity characteristics of a referenced material/element, wherein such shape-memory and/or superelasticity characteristics can relate to the tendency and/or ability of the subject material/element to deform when an external force is applied and return to the undeformed shape when the force is removed; as used herein, a material/element that includes shape memory can be understood to refer the shape memory effect and/or super elasticity. For example, a material/element that includes shape memory properties/characteristics (also referred to as “a shape memory element”) is configured to undergo deformation due to an external force/stress and return to its undeformed shape upon removal of the external force/stress, in some cases by changing the temperature of the material/element, and in other cases without changing the temperature of the material/element. To illustrate, a device with shape memory can include a biased/default shape/form, wherein the device is configured to be compressed, expanded, or otherwise deform when an external force is applied and configured to return to the biased/default shape/form when the external force is removed.

The stent segment 600 may be considered an oval stent segment with respect to the shape of the axial cross-section thereof, as shown in FIG. 6D. The term “oval” is used herein according to its broad and ordinary meaning and may be used substantially interchangeably with the term “ellipse” and/or “oblong,” which terms are likewise used according to their broad and ordinary meanings. The term “oval” may be used herein to refer to any non-circular closed curve having major and minor axes, the major axis being greater than the minor axis. With respect to “oval”-shaped stent segments disclosed herein, such stents may have relatively flatter minor-axis sidewalls (compared to curved major-axis end walls; e.g., wall segments 62), wherein the sidewalls may bow radially outward, and/or may be deflected/curved radially inward so as to produce external concavity and internal convexity in such sidewalls (e.g., forming a peanut-shaped stent). Major-axis walls of an oval stent as described herein may be considered wall portions of a stent that are intersected by a major axis of the stent that runs through an axial center of the stent. Minor-axis walls of such oval stents may be considered wall portions that are intersected by a minor axis of the stent that runs through the axial center of the stent. Example stents of the present disclosure may be considered to have an oval shape whether or not the shape thereof is definable by an algebraic curve. Example stents of the present disclosure may be considered oval stents when the wall(s) of the stent in an axial-cross-sectional perspective form(s) a closed or open curve, in a plane Pₛ, that is non-circular; one or more segments/areas thereof may resemble the outline of a portion of an egg. Oval stents of the present disclosure may include either one or two axes of symmetry of an ellipse, such as the illustrated major Aₘₐⱼ and minor Aₘᵢₙ axes. The axial cross-section of some examples of oval stents of the present disclosure may resemble the union of two semicircles on opposite sides of a rectangle, providing a shape evoking the likeness of a speed skating rink or an athletics track. In some contexts, the oval stent segment 600 may be referred to as a “stadium”-shaped stent, or an elongated oval.

The stent frame 631 comprises stent wall(s) defining an elongated tubular structure having a first axial end 621a with a first opening 622a. The tubular structure may further comprise a second axial end 621b with a second opening 622b, wherein the lumen/channel 649 extends between the first opening 622a and the second opening 622b, traversing the length L of the stent segment 600. The frame 631 and/or wall(s) thereof may comprise an open-cell structure adapted to be expanded to secure the stent segment 600 to a blood vessel internal (or external) wall, such as through endothelialization of the frame 631 to the vessel tissue over time.

In the oval configuration shown in FIGS. 6A-6D, the stent segment 600 may have a cross-sectional area having a major/long-axis diameter dₘₐⱼ that is substantially larger than the minor/short-axis diameter dₘᵢₙ. For example, the major-axis diameter/dimension dₘₐⱼ may advantageously be at least twice as long as the minor-axis diameter/dimension dₘᵢₙ, or even 3, 4, 6, 6, or 7 times greater. The stent segment 600 may be configured to increase compliance of a blood vessel though constant or near-constant pressure at one or more points along a perimeter/circumference of the blood vessel that causes a change in the perimeter geometry of the vessel. For example, the blood vessel may be changed and/or moved from a non-circular/less-circular shape to a circular/more-circular shape.

The stent frame wall(s) 631 may be at least partially composed of struts 638 and/or openings/cells 635 between the struts 638. The dimensions and/or shape of the stent segment 600 may vary based on the particular application and/or target implantation anatomy. For example, the length L of the individual stent segment 600, or the collective length of longitudinally offset/arranged stent segments in a modular configuration as described herein, may be selected to extend over all or a portion of an identified non-compliant length of a target blood vessel. The stent major axis dₘₐⱼ and minor axis dₘᵢₙ, when averaged, may be approximately equal to the diameter of the native blood vessel. For example, for a stent configured for deployment in an aorta, the length L and/or the total length of a modular stent implant of which the stent segment 600 is a component, may be between 1-30 cm. In the biased oval/diastolic configuration, the major axis dₘₐⱼ; may be between 1-4 cm (or larger/smaller depending on the particular anatomy), and the minor axis dₘᵢₙ may be between 20-50 percent of the major axis dₘₐⱼ. However, other sizes and/or shapes are also within the scope of this disclosure.

FIG. 7 shows an axial view of the stent segment 600 of FIGS. 6A-6D deployed within a blood vessel 61 in accordance with one or more examples. The stent 600 may be biased toward the illustrated oval and/or other non-circular relaxed/diastolic configuration (shown in solid-line), and may, when subjected to mechanical forces associated with high luminal pressure, be configured to responsively transform to a more-circular systolic configuration (shown in dashed-line) such that the minor axis dₘᵢₙ approaches, and may equal, the major axis dₘₐⱼ. As with any of the examples disclosed herein, the stent segment 600 can be configured to deflect from the oval shape to a more-circular shape in the presence of threshold blood pressure levels greater than 80 mmHg, such as blood pressure levels greater than 90 mmHg (e.g., between 90-120 mmHg).

The stent 600 may be configured to be percutaneously delivered to the blood vessel 61 in a compressed delivery configuration. Once within the blood vessel lumen at the target deployment site, the stent segment 600 may be configured to be radially expanded into direct surface contact with the blood vessel wall (e.g., the inner wall of an aorta segment). In some examples, the stent segment 600 may be configured to be expanded such that the perimeter of the stent frame 631 approximates and/or exceeds a perimeter of the blood vessel portion where the stent segment 600 is implanted, at least immediately prior to deployment/expansion of the stent segment. Placement of the stent segment 600 in the blood vessel may cause at least slight stretching in the blood vessel wall 61, such as due to pressure at the major-axis ends 627 against the blood vessel wall 62. In some cases, a stent configured to expand to a greater perimeter than the native blood vessel may provide improved traction and/or resistance to migration within the blood vessel. Implanting a stent that has a perimeter approximate to and/or slightly greater than the blood vessel perimeter may increase positive engagement with the blood vessel wall and/or maximize a compliance effect. The stent wall and/or a portion thereof may be configured to be endothelialized to the blood vessel wall. The configuration of the stent segment 600 in the oval shape can cause the blood vessel wall 61 to assume a more oval shape to match the shape of the stent 600. However, depending on the relative size of the stent 600 to the vessel 61, the blood vessel 61 may not necessarily conform exactly to the circumference and/or shape of the stent 600, and gap(s) 68 may be present and/or form between the frame 631 and the blood vessel wall 61 as the luminal pressure increases and pushes the vessel side walls away from the frame sidewall 625.

When implanted in a blood vessel, the patient physiology may respond to the stent 600 as a foreign object. For example, macrophages can accumulate around the stent, and nearby smooth muscle cells can proliferate to cover the stent. Over time, a new endothelial layer can form over the stent, which can inhibit clot formation. In addition to preventing thrombus formation, endothelialization can enhance the ability of the stent to reshape the target blood vessel by strengthening the physical coupling between the stent and the blood vessel, thereby reducing the presence of gaps forming between the stent and blood vessel wall when the stent walls pull away from the blood vessel wall as the stent reshapes to an oval/non-circular shape.

The stent frame 631 may or may not be covered, internally and/or externally, with an at least partially fluid-tight covering. For examples in which the stent frame 631 is not covered by a fluid-tight covering, the cells 635 of the frame 631 can provide openings in the frame 631 that allow blood in the blood vessel 61 to transfer pressure through the frame 631, to thereby load the inner diameter/surface of the blood vessel with a force resulting from increases in the luminal blood pressure as the heart beats. For example, luminal pressure forces against the blood vessel wall increase the hoop stress on the blood vessel, which may force the blood vessel, and with it the stent frame 631, to assume a more-circular shape. The resulting hoop stress from luminal pressure increase can exert radially-outward force along the blood vessel's inner circumference, such stresses/forces being tensile in nature, which can tend to cause the blood vessel to increase in diameter. The blood pressure force on the blood vessel wall and resulting inward deflection of the blood vessel wall (due to outward deflection of the vessel wall portions in the area of the minor axis Aₘᵢₙ) at the major-axis-ends 627 of the stent segment 600 may cause inward deflection of the ends 627 of the stent 600 to form a desired geometric change to a more-circular shape 600′ (shown in dashed line) of the stent and the blood vessel 61. The transition of the stent from oval to the more-circular stent shape 600′ (shown in dashed-line in FIG. 7) causes energy to be stored in the stent frame 631 (e.g., in the elasticity and/or shape memory thereof), such that energy is returned to the blood vessel walls, and therefore to the blood circulation within the blood vessel segment, when the frame transitions back to the oval shape 600 as pressure decreases.

The natural cross-sectional shape of the aorta (and other blood vessels) may generally be circular. With the oval stent segment 600 pushing outward to the oval configuration, the wall portions 63 may be pulled/drawn at least partially towards an axial center Aᵥ of the blood vessel 61 and/or towards each other in a manner as to cause the blood vessel 61 to form a non-circular shape, such as the oval shape shown in FIG. 7.

For examples in which the stent frame 631 is covered internally and/or externally by a fluid-tight covering (not shown in FIG. 7 for visual clarity), the openings of the cells 635 of the frame 631 may be closed to pass-through fluid and prevent blood in the blood vessel 61 and within the channel 649 of the stent segment 600 from transferring pressure through the frame 631. Therefore, intraluminal pressure within the flow channel 649 loads against the covering and frame 631 (rather than directly against the blood vessel walls 63) to provoke reshaping thereof. The blood-pressure-induced force against the covering and/or stent frame 631 can increase the hoop stress on the frame 631 and/or covering, which may force the frame 631, and with it the blood vessel 61, to assume a more-circular shape. Unlike with bare-frame stents that do not include fluid-tight coverings, where a covering is present, the endoluminal pressure forces in the channel 649 of the covered stent segment 600 are distributed over/against the covering and/or frame 631 to directly reshape the stent frame, rather than indirectly reshaping the stent segment 600 through force on a finite number of end contact/pressure points of the frame 631. As the pressure in the channel 649 increases (e.g., in connection with the systolic phase of the cardiac cycle), the plastically-deformable nature of the stent frame 631 allows for the sidewalls 625 of the frame 631 to be pushed outward along with the shortening of the stent segment 600 in the major axis dimension Aₘₐⱼ. When the sidewalls 625 are deflected outward, the channel 649 assumes a more circular cross-sectional shape.

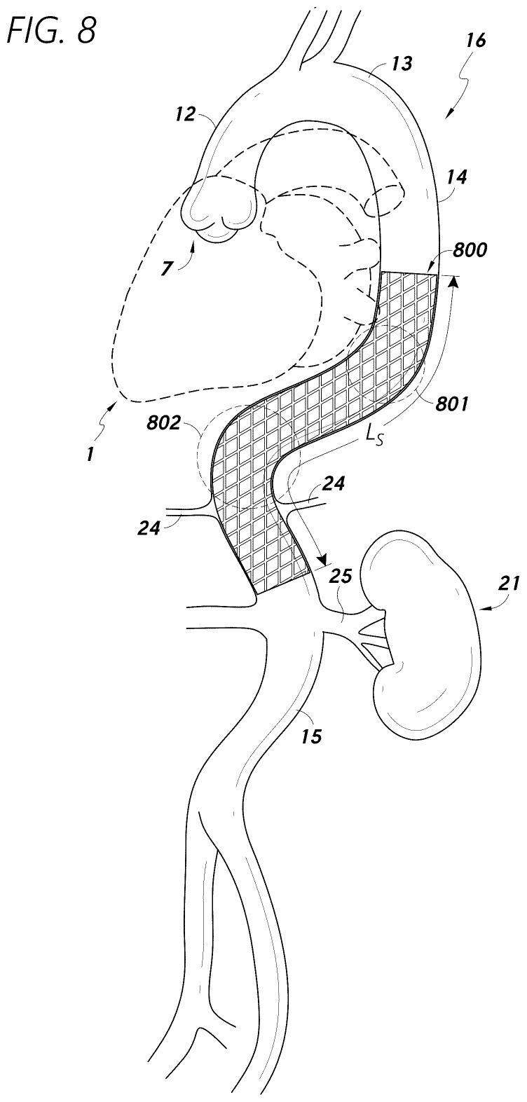

FIG. 8 shows a stent 800 spanning a curved blood vessel segment in accordance with one or more examples. The stent 800 have a circular or non-circular (e.g., oval, peanut, etc.) shape with respect to at least a portion of a length thereof. In some cases, it may be desirable for a stent to span a relatively long segment of a target blood vessel in order to achieve the desired therapeutic effect. In the example of FIG. 8, the stent 800 is implanted in a tortuous segment of the aorta 16, wherein the stent 800 spans one or more bends or curves (e.g., 801, 802) in the blood vessel 16. Such length Lₛ of the stent 800 may pass over and/or at least partially occlude one or more side branches 24 of the aorta or other target blood vessel. For example, the blood vessel segment in which the stent is implanted may include certain arterial branches, such as one or more of the phrenic arteries, suprarenal arteries, the celiac trunk, the superior mesenteric artery, the renal arteries, the lumbar arteries, and/or other branches. In examples in which the stent frame is covered with a covering on an inner and/or outer diameter thereof, such covering may occlude blood flow passage into side branches.

While a unitary, straight stent (e.g., oval stent) can be implanted in relatively linear segments of a patient's vasculature, if the target blood vessel segment is highly bent/tortuous, as shown in FIG. 8, a single straight and relatively rigid stent, as shown in FIG. 8, may be unsuitable or undesirable. As an alternative to the relatively long unitary stent shown in FIG. 8, implantation of a series of relatively-shorter stent segments can address challenges associated with implanting stent implants in tortuous blood vessel segments, thereby allowing such tortuous segments to be treated with compliance-enhancing stent implant devices (e.g., oval stent implants).

In some implementations, the present disclosure provides modularity for a total vascular length treated by a stent implant device and/or assembly using an implant comprising a plurality of stent segments positioned in series, wherein such stent segments may comprise oval or other non-circular stents/segments. Modular stent implants of the present disclosure may facilitate the implantation of oval stents segments in a relatively tortuous portion of a patient's vasculature. Modular stent implants of the present disclosure may further serve to preserve blood flow into side branches for vascular segments treated by compliance-enhancing stents or other stent devices. Implanting modular stent implants comprising a plurality of stent segments arranged in series within the target blood vessel can allow for customization of the length spanned by the stent implant. For example, the number of stent segments implemented may be selected and/or constructed to a specific length conforming to a specific blood vessel segment of the patient.

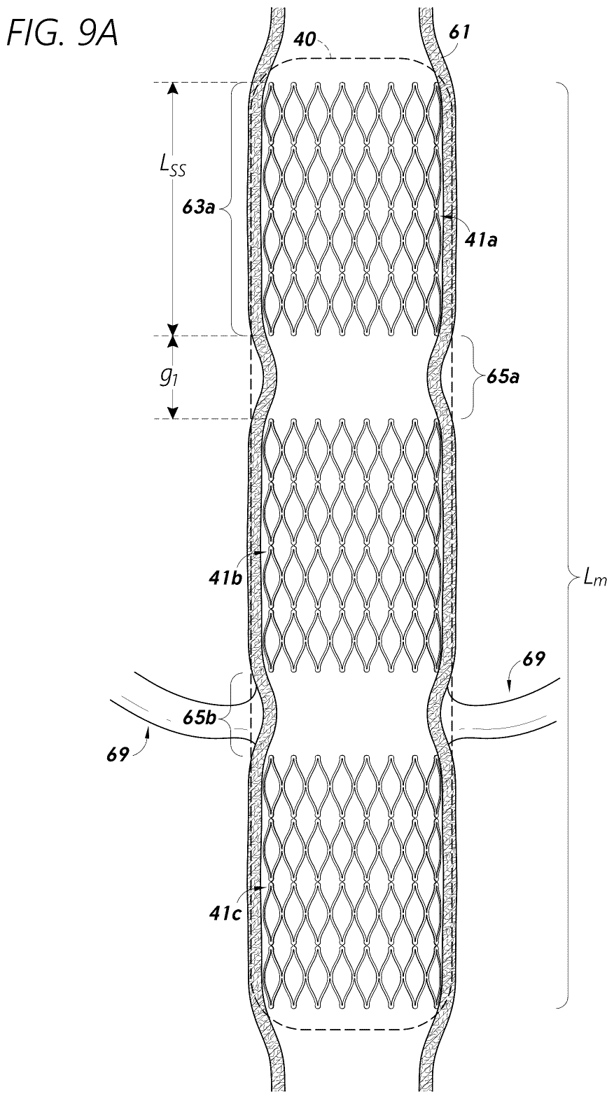

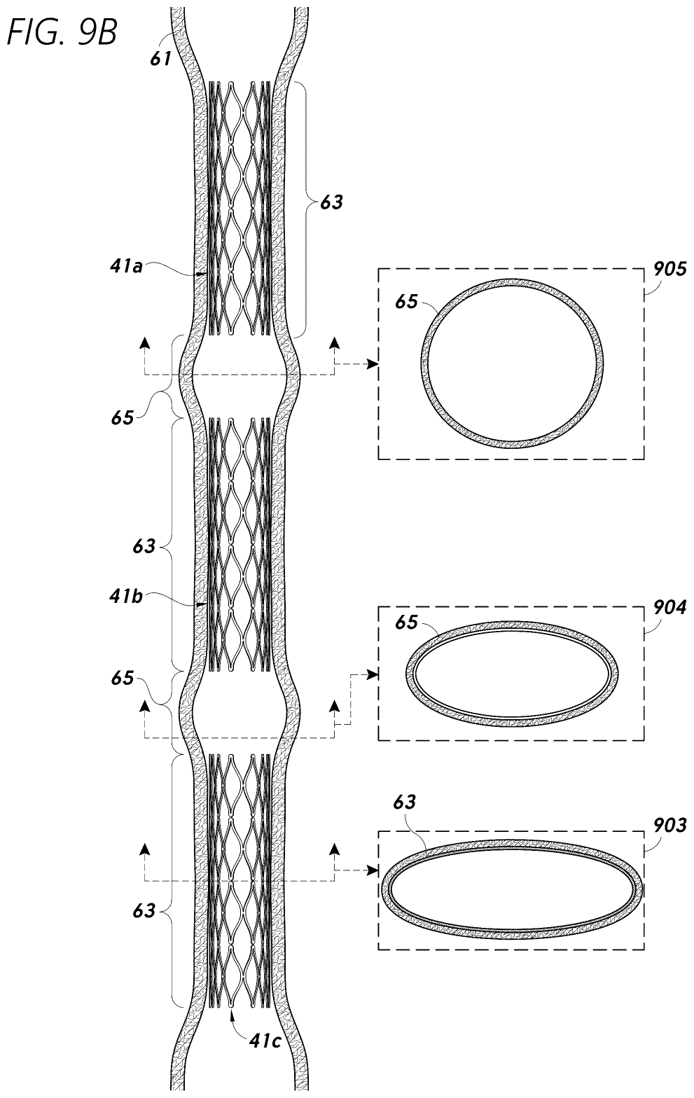

FIGS. 9A and 9B show cutaway views of modular stent segments 41 of a modular stent implant 40 placed in series within a blood vessel segment 61 in accordance with one or more examples. In some implementations, a plurality of relatively short non-circular/oval stent segments are serially implanted, wherein such segments 41 may or may not be mechanically coupled to one another. The number of stent segments may be chosen according to the patient-specific anatomy. The serial arrangement of the relatively-shorter stent segments may provide a flexible alternative to a single unitary stent as shown in FIG. 8.

The stent segments 41 may comprise bare-metal or covered stent segments having a biased oval or other non-circular cross-sectional shape in a free state thereof. The stent segments 41 may be implantable in any target blood vessel, such as the aorta, so as to force the target blood vessel to assume a more oval shape under relatively low-pressure conditions (e.g., diastole), wherein increased blood pressure in the target blood vessel (e.g., during systole) forces the blood vessel to assume a more circular shape. Such cyclical reshaping, as described in detail herein, can restore and/or increase compliance in the target blood vessel, which may be a relatively non-compliant or less-compliant blood vessel segment.

The use of the plurality of short stent segments 41 can allow for the implant 40 to be implanted in a modular manner, wherein the number of stent segments implemented and/or the space between adjacent stent segments can determine the overall length Lₘ of the vascular segment treated by the implant 40. In some implementations, the stent segments 211 may have a length Lₛₛ of between 3-5 cm. In some implementations, the patient's clinical/physiological measurements may be acquired in real time during the implantation process, wherein oval stent segments may be gradually added/deployed until the desired improvement of such measurements is achieved/apparent. The various segments 41 of the implant 40 can be a similarly or differently formed/configured. For example, in some implementations, the various stent segments 41 can have different geometries with respect to length, diameter, and/or other aspects thereof. Furthermore, different stent segments 41 may comprise different materials, such that a given stent segment may be adapted for the specific anatomical region in which it is implanted. For example, different stent segments 41 of the implant assembly 40 may be more or less rigid than others. Using a modular stent implant having stent segments with different dimensions and/or configurations can provide for more precise blood vessel diameter adjustment. That is, there may be an optimal diameter for each of the stent segments 41 with respect to the respective blood vessel segment in which it is implanted. For example, the stent segments 41 may have different circumference and/or diameter dimensions based on different circumferences/diameters of the vessel in different areas thereof, which may provide enhanced performance for the stent implant 40.

In some implementations, the stent segments 41 may be covered on an outer diameter, inner diameter, or both, with a covering. Such covering may be at least partially fluid-blocking/tight. The use of coverings on oval or other non-circular stent segments may be desirable as a means to increase in luminal blood pressure forces against the frame of the stent segments to cause reshaping thereof, due to the coupling between the frame and the covering. Where covered stent segments are used, if the stent segment passes over a side branch 69 of the blood vessel, the covering may block flow and into the side branch. Therefore, in some implementations, the stent segments 41 may be arranged such that any side branches 69 from the target blood vessel 61 are positioned in gaps 65 between adjacent stent segments. Therefore, utilizing a plurality of relatively shorter stent segments as in FIGS. 9A and 9B can mitigate the risk of blood flow occlusion by leaving side branches exposed in the spaces 65 between adjacent stent segments 41. The stent segments 41 can be separated from one another by any suitable or desirable distances g₁, including varying distances between separate pairs of adjacent stent segments in a single assembly/implant. The gap distance(s) g₁ can be any distance between approximately 1 mm and 5 cm, wherein the chosen distance(s) can be based at least in part on the particular native anatomy, tortuosity, blood vessel wall calcification, presence of side branches, and/or other factors. Example gap distances g₁ between adjacent stents/segments 41 can be between 0-1 mm, 1-2 mm, 2-5 mm, 5-10 mm, 1-1.5 cm, 1.5-2 cm, 2-3 cm, 3-4 cm, 4-5 cm, or greater than 5 cm.

The longitudinal distance g₁ between adjacent oval stent segments 41 may be selected to provide a spacing that produces an at least partially oval shape in the blood vessel segments 65 disposed in the gaps between stent segments 41. That is, whereas the frames of the stent segments 41 produce the desired reshaping of the blood vessel segments 63 with which they are aligned through direct contact therewith, the blood vessel segments/portions 65 in the gaps between stent segments 41 may not have direct reshaping due to the absence of the stent frames in contact with such vessel walls. Therefore, in such blood vessel segments 65, the reshaping thereof, if any, may be a result of the reshaping of the adjacent blood vessel portions 63 that are directly reshaped by the stent segments 41. As a result, as shown in FIG. 9B, the blood vessel segments 63 aligned with stent segments 41 may be reshaped to an oval shape in low-pressure conditions, as shown in axial image 903. The segments of the blood vessel disposed at the axial midpoint of the gap 65 between adjacent blood vessel segments, as shown in image 905, may have more circular shapes, wherein the shape of the blood vessel in the segments 65 may become more oval moving closer in distance to the ends of the stent frames 41. That is, the degree to which the blood vessel 61 may be reshaped to the oval shape may be proportional to the distance from a given stent frame. Therefore, in areas relatively near to the ends of the stent frame, as shown in axial image 904, the reshaping of the blood vessel may be greater than with respect to the mid-gap area 905, but less than in the areas aligned with the stent frames 903/63. Therefore, even blood vessel segments 65 not covered by stent segments 41 may benefit from a reshaping effect that can improve blood flow through the target blood vessel. Such functionality can effectively elongate the portion of the target blood vessel influenced by the transitioning stent segments, even beyond the length Lₘ of the implant 40. Therefore, modular stent implants of the present disclosure can advantageously provide a flow-enhancing effect for relatively long blood vessel segments, such as in portions of the abdominal and/or thoracic aorta. The gap areas 65 of the blood vessel, although they may tend to reshape to an oval shape to a lesser extent than the blood vessel segments 63 contacting the stent segments 41, the gap segments 65 may nevertheless be reshaped to some degree and provide a contribution to compliance enhancement.

Specific gap distances g₁ between stent segments 41 may be implemented to provide the desired reshaping of the blood vessel 61 in the stent-less/free blood vessel segments 65, while maximizing the overall length Lₘ of the implant 40. The presence of the gaps 65 between adjacent stent segments 65 may advantageously allow for blood flow provision to side branches 69, as described in detail herein. In some implementations, the stent implant 40 is implemented as a single/unitary stent frame with cover segments that correspond to the illustrated stent segments in FIGS. 9A and 9B. That is, while the stent frame may span substantially the entire length Lₘ of the implant, the covering may include gaps that service the side branches 69 to allow flow through the frame into the side branches. Such implementations may allow for control over unwanted blocking of the blood supply to peripheral arteries.

The stent segments 41 may include internal or external coverings, such as fabric and/or polymer, fluid-tight covers. In some implementations, the coverings may be disposed on an outer diameter of the stent segment frames 41 in areas along the major-axis sides/walls of the stent segments to distribute the contact load between the blood vessel 61 and the stent segment frames and/or to protect the blood vessel inner wall from abrasion or other damage from the stent segments due to the concentrated pressure/force at the major-axis ends/walls. While the stent segments 41 are illustrated as oval stents, it should be understood that any or all of the collection of modular stent segments 41 of the implant 40 may be circular stent segments.

Rather than using a single elongated oval stent, some examples presented herein provide for segmented/modular stents with sub-parts implanted in a series arrangement. Adjustments in the length of the collective implant can be implemented to accommodate the particular patient anatomy. For example, relatively tall patients with relatively long aortas or other target blood vessels can be treated using implants with relatively higher numbers of stent segments. The effect of the implanted stent segments may be monitored in real time, such that the surgeon may cease stent segment introduction/deployment at the point where the desired therapeutic effect is realized. Although the stent segments of modular stent examples disclosed herein are described as ovalized blood-vessel-reshaping stents, it should be understood that any type of compliance-enhancing stent/structure may be implemented in a modular fashion as disclosed herein within the scope of the present disclosure. For example, the segments of the modular devices disclosed herein may comprise elastically stretchable structures configured to expand and contract in a cyclical manner in response to changing pressure conditions so as to increase compliance.

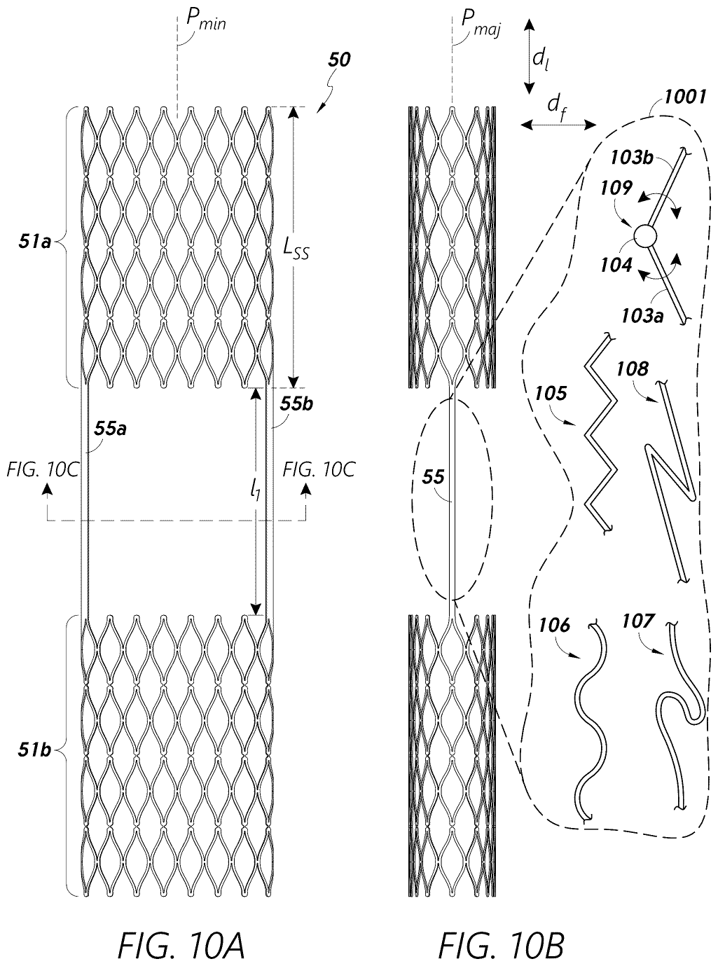

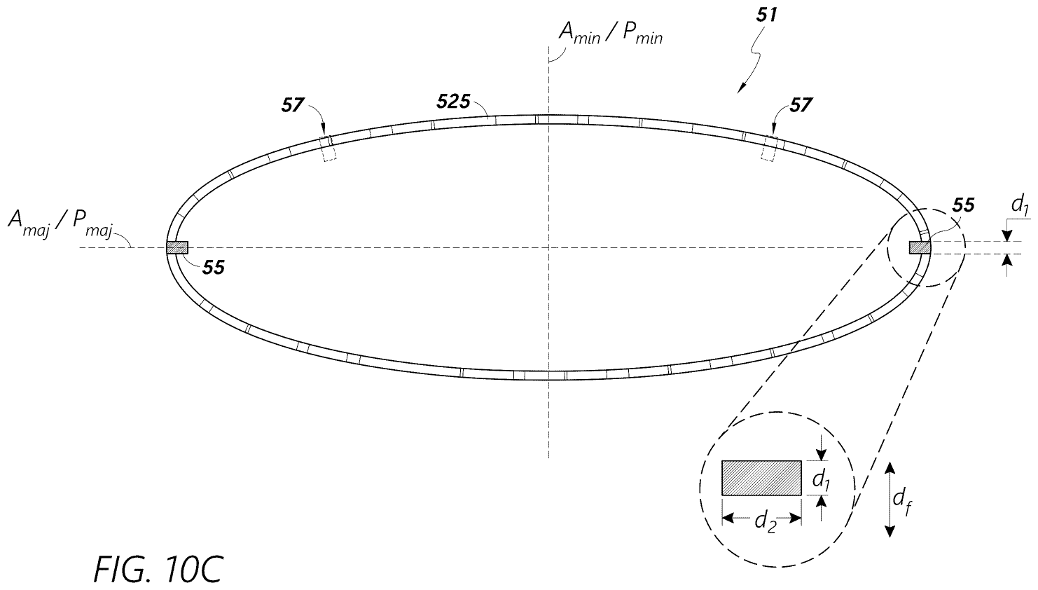

Assemblies/collections of stent segments implemented as a modular stent implant device according to aspects of the present disclosure can be implemented with segment-to-segment connectors/links, or without connections, as shown in FIGS. 9A and 9B. FIG. 10A shows a view of a minor-axis side of a modular stent 50 including non-circular stent segments 51 coupled by link/connector features 55 in accordance with one or more examples. FIG. 10B shows a view of a major-axis side of the modular stent 50 of FIG. 10A in accordance with one or more examples. FIG. 10C shows an axial view between the stent segments 51 of the modular stent 50 of FIGS. 10A and 10B.

As with other examples of the present disclosure, although only two stent segments are shown in FIGS. 10A and 10B, it should be understood that the implant device shown may have any number stent segments, each adjacent pair of which may be coupled by arm/connectors. The connectors/links 55 can provide various functions and/or benefits for the implant device 50. For example, the connectors/links 55 may help to align the various stent segments 51 of the modular stent 50. That is, as described in detail herein, the stent segments 51 may have non-circular cross-sectional shapes, without rotational symmetry; the cross-sectional shape of the stent segments may have major-axis and/or minor-axis ends/sides. In accordance with some procedures, it may be desirable to implant stent segments 51 of the modular stent implant 50 such that the major and/or minor axes thereof, respectively, are aligned. That is, the major axes of the stent segments may advantageously be aligned and the minor axes may likewise be aligned. Such alignments may reduce wear and tear on the anatomy of the blood vessel and/or may increase the efficiency/efficacy of the reshaping of the blood vessel by the stent segments and/or connectors/arms 55. The linking arms 55 can be configured to bend in a dimension parallel with a minor axis/plane of the stent segments 51 and/or resist deflection in a dimension parallel with a major axis/plane of the stent segments 51. The connecting arms 55 may be integrated with the stent frames, as shown.

In some implementations, it may be desirable to have stent segments of a modular stent rotationally offset from one another by a known angular offset. The segment connectors/links of such a modular stent can be utilized to force the desired rotational offset between adjacent stent segments. For example, graduated rotational offsetting of successive stent segments of a modular stent comprising three or more stent segments may promote desirable flow dynamics, such as funneling effects or other flow dynamics to facilitate/increase flow during at least some cardiac phases.

In some implementations, it may be desirable to orient oval/non-circular stent segments such that a plane Pₘₐⱼ associated with a major axis Aₘₐⱼ of the stent segment along a length thereof is aligned with the frontal/coronal plane of the patient. That is, the major axis plane Pₘₐⱼ may advantageously be most closely angularly aligned with the frontal/coronal plane compared to the sagittal and transverse planes when the modular stent 50 is implanted in the aorta. For reference, the sagittal plane may refer to a plane that divides the body into the left and right sections, whereas the coronal/frontal plane and the transverse plane divide the body into front/anterior and back/posterior sections, and upper/superior and lower/inferior sections, respectively. The alignment of the major axis Aₘₐⱼ as described, such that the minor axis walls 525 of the stent face forward and backward with respect to the patient orientation can advantageously facilitate the spanning of modular stents in accordance with aspects of the present disclosure through a curved blood (e.g., aortic) vessel segment that curves primarily in the forward/backward dimension, as can occur with respect to the thoracic and/or abdominal aorta (see FIG. 1B). That is, oval stent segments of modular stents of the present disclosure, and segment-to-segment connectors/links of modular stents disclosed here, can be configured to more easily curve/deflect in the minor-axis dimension of the implant due to the smaller minor-axis diameter compared to the major-axis diameter. Modular stent linking connector/arm structures can advantageously facilitate the alignment of adjacent stent segments to accommodate curving/bending of the implant along a tortuous blood vessel segment, wherein the minor axis of the stent segment(s) is aligned more closely with the direction of curvature of the aorta or other target blood vessel compared to the major axis of the stent segment(s).

As described above, for modular stent implants comprising a plurality of stent segments positioned with gaps therebetween, the inherent shape of end portions of adjacent stent segments can cause the blood vessel portion in the gap between adjacent stent segments to deform to at least partially assume the shape of the stent segments, thereby providing a virtual reshaping effect on the unstented gap portions of the target blood vessel that mimics, at least in part, the reshaping of the blood vessel in the area thereof overlapping the stent segments. However, as demonstrated above in connection with FIG. 9B, the degree to which the blood vessel in the gap segments is reshaped may be substantially less than the degree of reshaping in the stented blood vessel segments in some instances, depending at least in part on the distant spanned by the gap between adjacent stent segments. The decreased amount of blood vessel reshaping in the gap segments can be considered a waist effect, where the blood vessel rounds-out to some degree towards the middle of the gap segment.

In some implementations, modular stents of the present disclosure that comprise segment-to-segment connecting struts/arms/links can produce a greater degree of blood vessel reshaping in the gap areas between adjacent stent segments compared to modular stents that do not include connecting arms/struts. With linking struts/arms connecting adjacent stent segments on major axis ends of the stent frames, as in the illustrated example of FIGS. 10A-10C, such struts/links can serve to increase the reshaping of the blood vessel in the gap areas by stretching out the blood vessel along the major axis ends of the modular stent implant in the gap areas.

Although connecting links/arms/struts of modular stent implants of the present disclosure may be disposed at any position around the perimeter/circumference of the stent segments, positioning one or two connecting arms/struts on major axis ends/sides of the oval/non-circular stent segments can advantageously serve to flank the major axis ends/sides of the stent segments in a manner as to promote the stretching-out of the target blood vessel and elongation thereof in the major-axis dimension, thereby conforming more closely to the reshaping effect of the stent segments on the blood vessel in the areas overlapping therewith. Therefore, due to the connecting arms/struts 55, the gap segments of the target blood vessel between adjacent stent segments can be reshaped to a relatively flatter, more-oval shape, and a more consistent shape, along the length of the modular stent implant. Furthermore, the presence of the connecting arms/struts may allow for the gap between adjacent stent segments to be a greater distance, while still promoting desired compliance-enhancing blood vessel reshaping compared to implementations of modular stent implants that do not include connecting links/struts.

Flexure characteristics of the connecting arms/struts 55 may facilitate bending of the implant 50 around curves in a target blood vessel. For example, the connecting arms/struts 55 may be designed such that they resist radial inward bowing/deflection, which would potentially reduce the efficacy of the connecting struts/arms with respect to the blood vessel reshaping in the gap blood vessel area between the stent segments. In some implementations, as shown in FIG. 10C, the struts may have a radial dimension d₂ with respect to the axis of the stent segment that is greater than a circumferential dimension d₁, thereby producing connecting arms/struts 55 that are more inclined to bend/curve in the minor-axis dimension Aₘᵢₙ than in the major-axis dimension Aₘₐⱼ. That is, the greatest cross-sectional dimension of the struts/arms 55 may be generally orthogonal to the stent frame. With the connector arms/struts 55 optionally having a greater radial dimension d₂ than circumferential dimension d₁, lateral bending of the arms/struts 55 may correspond to bending generally in parallel with the minor axis Aₘᵢₙ, whereas vertical bending, which is impeded by the geometry of strut/arm, is generally in parallel with the major axis Aₘₐⱼ.

In some implementations, the stent frames of the stent segments 51 and the connecting arm/struts 55 may be formed by laser-cutting, stamping, or otherwise forming a sheet or tube of metal or other at least partially rigid material. That is, the struts 55 may be a unitary form with the stent frames of the stent segments 51. Alternatively, the connecting struts/links 55 may comprise separate components that may be coupled to the stent frames in some manner, such as by welding or other attachment means/mechanism. That is, the structure of the modular stent implant 50 may be a unitary structure or a joint structure. In some implementations, the struts/arms 55 comprise a thicker cross-section than the stent frame portions 51 to provide strength/integrity of the struts 55 to hold and/or reshape blood vessel walls.

In some implementations, the struts/arms 55 may be positioned circumferentially on the stent segments at positions 57 that are both on a common diametrical side/half of the stent segments, such that they are positioned between the major axis ends and the minor axis Aₘᵢₙ on a common side of the major-axis plane Pₘₐⱼ, as shown as the alternative dashed features 57 in FIG. 10C. Such placement may be on a side of the stent segments that is positioned forward/anterior when deployed in a thoracic or abdominal portion of the aorta to increase the distance between the struts 55 and posteriorly-positioned side branches, such as peripheral arterial vessels that supply the spinal cord or other anatomy. Such positioning 57 as in FIG. 10C may produce approximately 90° degrees of angular separation between the struts/arms 57 and the posterior/dorsal side branches.

In FIG. 10A, the arms/struts 55 are shown as having a longitudinally-straight shape. However, as shown in the detail 1001 of FIG. 10B, the connecting struts/links 55 can have any suitable or desirable shape, including the examples shown in the detail 1001. Example non-limiting strut designs shown in detail 1001 include wavy designs (e.g., arm 106, arm 107; arm 107 has an ‘S’ shape that curves and doubles-back, longitudinally in a longitudinally-overlapping manner), zig-zag designs/shapes (e.g., arm 105, arm 108; arm 108 has a ‘Z’ shape that is longitudinally overlapping), and hinged designs (e.g., arm 109). The shape of the struts/arms 55 may advantageously facilitate deflection of the major-axis plane of the implant 50 to provide curvature in the minor-axis dimension. In some implementations, the struts/arms 55 are designed with a natural shape that bows radially outwardly to some degree to resist inward deflection of the struts when the implant assembly is deployed, thereby increasing the efficacy of the reshaping functionality of the struts/arms 55.

In some implementations, the midspan of the connecting struts 55 has a curved and/or zig-zag shape that allows the strut to flex and/or curve in at least one dimension. The shape/design of the struts 55 may provide transverse stability to resist radial deflection, while having reduced resistance to bending/flexing in the dimension d_{f} shown in FIG. 10B. The struts 55 may be relatively flatter in the direction of flex thereof, wherein the flatness of the struts may facilitate the flexing in the desired dimension, while impeding flexing in the orthogonal dimension (e.g., orthogonal with respect to the blood vessel surface). In some implementations, the profile of the connecting struts 55 is nonuniform over the length thereof. For example, the struts/arms may be relatively thinner in certain spots to facilitate flexing/bending in such areas/portions of the struts, whereas the struts may be more rigid and less prone to bending/flexing in areas thereof that a relatively wider. Such shaping of the connecting struts/links 55 can be implemented to provide preferential bending/flexing in the desired dimension and resist bending/flexing in the orthogonal dimension. For example, with respect to the orientation of FIG. 10B, the design of the struts 55 may facilitate bending in a dimension parallel with the plane of the page, while impeding bending/flexing in and out of the page.

In some implementations, the shape of the struts 55 may facilitate axial length flexibility in addition to bending/flexing as described above. For example, the curves/bends of the struts 55 may allow for the stretching or compression in the longitudinal/axial dimension d₁ to allow the surgeon the ability to customize the gap length between adjacent stent segments by manually or otherwise manipulating the shape/bend of the strut features. Therefore, the connecting struts/links 55 may be designed to be radially stiff for supporting vessel reshaping, while also allowing longitudinal flexibility to enable vessel conformability along the length of the target blood vessel segment. The wavy and/or zig-zag designs illustrated in detail 1001 may provide the ability to bend longitudinally and thereby adjust the length of the struts.

In some implementations, the connecting arms/struts 55 comprise one or more hinge features (e.g., hinge feature 104 of arm 109 in detail 1001) in the strut(s)/arm(s). For example, a dovetail joint or other hinge means or mechanism may be implemented to couple respective struts/arms 103 emanating from the adjacent stent segments, while allowing for bending/deflection at the hinge/joint. Any type of hinge mechanism/means may be implemented, such as one or more hooks, pins, apertures, rods, tabs, or the like. Such hinge means/mechanism may advantageously retain radial stiffness/rigidity in the connecting link/arm assembly, while allowing bending flexibility in the desired dimension. In some implementations, elastic covering or material is implemented as or over arms connecting adjacent stent segments, wherein such elastic component(s) allow for the desired flexing/bending of such coupling arm.

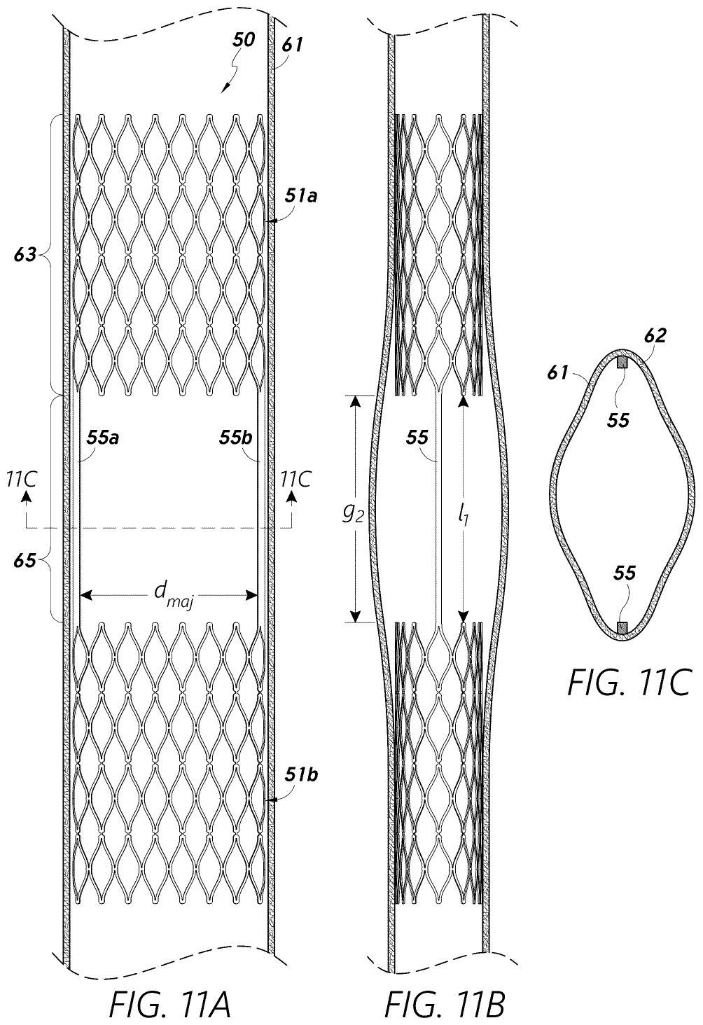

FIG. 11A shows a view of a minor-axis side of the modular stent 50 implanted in a blood vessel segment 61 in accordance with one or more examples. FIG. 11B shows a view of a major-axis side of the modular stent 50 implanted in the blood vessel segment 61 in accordance with one or more examples. FIG. 1I C shows an axial view between the stent segments 51 of the modular stent 50. With the modular stent 50 implanted in the blood vessel 61, the connecting arms/struts 55 may allow for relatively longer gap distance g₂ between adjacent stent segments 51, while still holding at least partially ovalized shape in the gap segment 65 of the blood vessel 61 that is spanned by the arms/struts 55. The arms/struts 55 may further hold the stent segments 51 at the desired distance longitudinally offset from one another, such that the implant 50 spans the desired length of the target blood vessel 61.

As shown in FIGS. 11A-11C, the arms/struts 55 hold the separate stent segments 51 in angular alignment, such that the major axis and minor axis of the stent segments are aligned from one stent segment to another or held at a known/desired angular/rotational offset. The arms/struts 55 may further provide a benefit with respect to delivery by clocking the stent segments 51 in a common alignment/orientation during delivery, such that the stent segments 51 do not migrate or become clocked out-of-plane with respect to one another. Alignment of stent segments 51 during delivery can facilitate proper delivery and deployment of the stent segments, such as by using certain delivery system instrumentation (e.g., pushers, couplers, etc.). With the oval stent segments 51 held in alignment by the arms/struts 55, the major axis of the stent segments 51 may expand in a common dimension, as shown in FIG. 11A, and the minor axis may contract and expand in a common dimension as shown in FIG. 11B. By utilizing the connecting struts/links 55 to align the stent segments 51, the risk of stress/damage to the target blood vessel may be reduced, as the stent segments 51 may tend to expand and contract along similar dimensions, thereby reducing pressure forces on the blood vessel wall.

The axial view of FIG. 11C shows the connecting arm/struts 55 stretching-out the blood vessel 61 in the gap segment 65 between the stent segments 51. The arms/struts 55, as shown, advantageously can increase the reshaping of the blood vessel 61 in the gap segments/areas 65, thereby increasing the reshaping effect of the implant 50. As the struts/arm 55 are positioned at distinct, finite positions around the perimeter of the stent segments 51, the reshaping effect thereof may not be entirely as effective as the reshaping in the segments 63 of the blood vessel that are directly contacted fully around the inner diameter thereof by the stent segments 51. However, compared to implementations in which no connecting arm/struts are provided, the ovalization of the blood vessel segment 65 can be to a greater degree with respect to examples that include connecting struts/arms as with the implant 50. By implementing the arms/struts 50 at opposite circumferential sides/areas of the stent segments, and particularly by associating the arm/struts 55 with the major-axis ends/sides of the stent segments 51, the reshaping effect of the struts/arms 55 may be maximized, at least with respect to two-arm examples. For example, by implementing the struts/arms 55 at the major-axis ends of the oval stent segments, the greatest diameter dₘₐⱼ of the stent segments may be forced in the gap areas 65 by the struts/arms 55, thereby increasing the reshaping of the blood vessel.

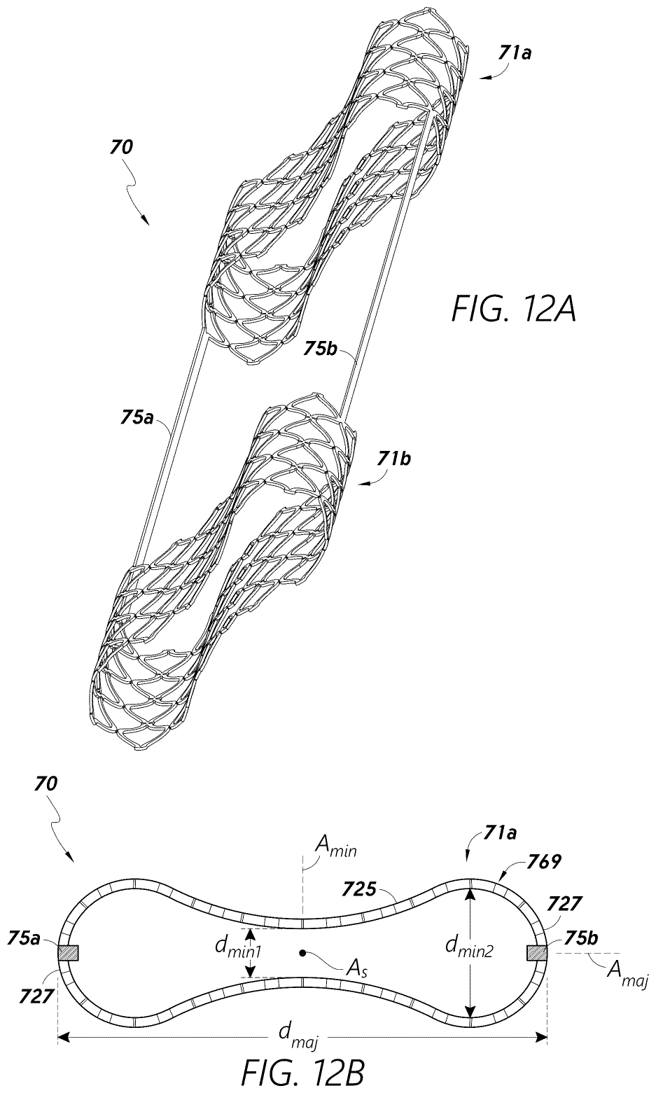

FIGS. 12A and 12B show perspective and axial views, respectively, of a modular stent 70 including peanut-shaped stent segments 71 coupled by link/connector features 75 in accordance with one or more examples. The stent segments 71 may represent example implementations of any of the non-circular/oval stent segments disclosed herein, or portion(s) thereof.

The shape of the stent segments 71 deviates from the stent segments 51 shown in FIGS. 10A-10C only in that the minor-axis (e.g., relatively flat and/or long) sidewalls 725 deflect to a greater degree towards the center/axis Aₛ of the stent segments 71 in a relaxed state. The resulting shape may resemble that of an hourglass and/or peanut shape with respect to the axial cross-section shown in FIG. 12B. The stent segments 71 may have a major axis diameter/dimension dₘₐⱼ that is greater than a minor axis diameter/dimension dₘᵢₙ₁, dₘᵢₙ₂ of the peanut cross-section.

In some examples, the stent segments 71 may be biased to a shape having a minor axis dimension dₘᵢₙ that is non-constant along the major axis Aₘₐⱼ dimension, which forms an externally-concave/internally-convex surface/form with respect to the minor-axis sidewalls 725. For example, the minor-axis sidewalls 725 may have a diameter dimension dₘᵢₙ₁ at a center thereof (with respect to the major axis dimension Aₘₐⱼ) that is less than the diameter/dimension dₘᵢₙ₂ at/towards the end portions of the minor-axis sidewalls 725. Non-circular stents of the present disclosure that have externally-concave/internally-convex minor-axis sidewalls as shown in FIGS. 12A and 12B are referred to herein as peanut-shaped stents; such peanut shape can be considered a variation of an oval, or oval-shaped, stent as described herein. The inwardly-deflected walls 725 can allow for a transition reshaping between peanut-shaped, to outwardly-bowed oval shape, and ultimately to circular/more-circular shape as luminal pressures are increased.

The stent segments 71 may be biased (such as being memory-set via a memory material such as nitinol) toward the illustrated peanut shape. With the sidewalls 725 deflected inwardly, as shown, the stent frame 71 may form bulging/bulbous portions 769 on either side of the minor axis Aₘᵢₙ, wherein such portions have a diameter/dimension dₘᵢₙ₂ that is greater than the shortened diameter dₘᵢₙ₁.

Once deployed within a blood vessel, during diastolic pressure, the stent segment sidewalls 725 may remain at least partially inwardly deflected or, due to blood pressure levels present (even during low-pressure conditions. e.g., diastole), the stent segments 71 and/or blood vessel may deform into a substantially oval, non-peanut shape. When subjected to the higher pressures of systole, the stent segments 71 and/or blood vessel may deform to an even more circular shape.

The stent segments 71 may or may not have coverings associated therewith. The terms “associated” and “associated with” are used herein according to their broad and ordinary meanings. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

The modular stent implant 70 may include any number of peanut-shaped stent segment 71, axially-adjacent ones of which may be coupled by connecting arms/struts 75 as described in detail throughout the present disclosure. The arms/struts 75 may connect to the stent segments 71 along any portion of the perimeter thereof, such as at major-axis end walls/points, as shown.

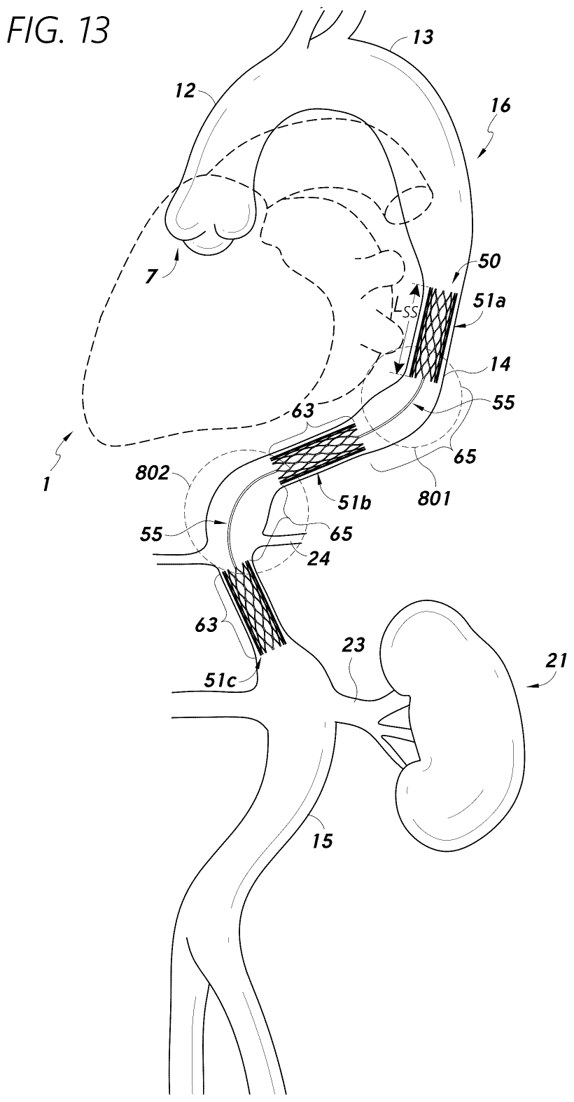

FIG. 13 shows a modular stent 50 implanted in a curved blood vessel segment in accordance with one or more examples. The stent implant 50 shown in FIG. 13 may be similar in one or more respects to any the modular stents shown and/or described herein, such as the modular stent of FIGS. 10A-10C.

The modular stent 50 may be implanted in the anatomy such that the struts 55 connecting the stent segments 51 are generally closer in position to sides of the arch/curve 801, 802 in the vascular anatomy spanned by the implant 50. As described above, the struts 55 may be associated with longer-axis ends of the non-circular stent segments 51. The struts 55 are configured to bend in the plane of the curve(s) (e.g., 801, 802) of the anatomy. Each adjacent pair of stent segments 51 may advantageously be coupled by two connecting link/struts 55. For implant devices implanted in relatively straight blood vessel segments, it may be suitable or desirable to implement connecting struts/arms 55 on both major and minor axes of the stent segments 51 to increase the blood vessel reshaping in the gap segments 65 between adjacent stent segments. However, with respect to deployment in a tortuous blood vessel segment as in FIG. 13, minor-axis sides/walls of the adjacent stent segments 51 may advantageously not have connecting struts connected thereto, such that the connecting struts 55 are limited to major-axis sides/end-walls of the respective stent segments 51.

The minimalistic structure of the connecting struts/arms 55 relative to the structure of the stent frames of the stent segments 51, particularly with respect implementations in which the stent segment frames are covered with cloth or polymer coverings, can provide flow gaps through which blood flow may service side branches 24 off of the main blood vessel in which the implant 50 is implanted. For example, the gaps 65 between stent segments can provide a space for intercostal artery side branches to receive blood flow from the aorta 16. Therefore, the connecting struts/arms 55 can advantageously provide desirable peripheral blood flow while increasing the vascular reshaping in the gap segments 65 between stent segments to thereby improve the efficacy of the implant device 50. Furthermore, the modular stent implant 50 may be substantially more flexible than a single unitary stent having a similar length, as shown in FIG. 8, due to the struts/arms 55 being more easily bent and/or more flexible than the stent frames of the stent segments 51 themselves, wherein the struts 55 can relatively easily bend through the curves 801, 802 of the target blood vessel. The gap distance(s) g₂ can be any distance between approximately 1 mm and 7 cm, wherein the chosen distance(s) can be based at least in part on the particular native anatomy, tortuosity, blood vessel wall calcification, presence of side branches, and/or other factors. Example gap distances between adjacent stents/segments 51 can include. Example gap distances g₂ between adjacent stents/segments 51 can be between 2-5 mm, 5-10 mm, 1-1.5 cm, 1.5-2 cm, 2-3 cm, or greater than 3 cm.

It may be desirable to position the connecting struts 55, e.g., with respect to the perimeter of the stent segments 51 and/or with respect to how the stent segments 51 are clocked/oriented within the target blood vessel, such that they do not pass over blood vessel side-branch ostia to reduce interference of the struts 55 with blood flow into side branches (e.g., arterial branches 24). However, in some implementations, the structure of the struts 55 may be sufficiently minimalistic with respect to the thickness and/or other dimension(s) thereof, such that even in cases where the struts 55 pass over the ostium of a side branch, blood flow may still be permitted into the side branch ostium around the connecting strut(s)/arm(s) 55. In some implementations, a modular stent implant in accordance with aspects of the present disclosure may be implanted at least partially within the area of the thoracic aorta 14 in a target blood vessel segment that includes one or more intercostal side branches. Such blood vessel side branches may generally be positioned on a posterior half of the aorta and generally angularly offset from the diametrical sides of the aorta, such that when the implant 50 is deployed with the major-axis ends of the stent segments disposed generally at the sides of the aortic vessel (e.g., side vertices in a plane that is within 45° of parallel with the frontal/coronal plane), wherein the connecting struts/arms 55 are associated with the major-axis ends/sides of the stent segments 51, the connecting arms/struts 55 may not overlap and/or interfere with the intercostal side-branch ostia.

The implant 50 may advantageously be implanted at such an orientation wherein the major-axis ends of the stent segments 51 and/or the connecting arms 55 are disposed within 10° of the sides of the curve(s) of the blood vessel 16. For example, the “sides” of a curve in the target blood vessel may correspond to areas of the blood vessel that are positioned farthest from the plane of the curve with respect to a given axial position. Therefore, in the example orientation of FIG. 13, wherein the curve 802 is in the plane of the page, the sides of the curve 802 and/or vessel 16 in such area may be considered the portions of the blood vessel cylinder/wall that would be farthest away from the plane of the page moving in in/out of the page in a three-dimensional representation. For example, such areas may correspond to the area in the center of the blood vessel with respect to the side profile of FIG. 13.

With the connecting struts/arms 55 positioned on the major-axis ends of the oval stent segments 51, the blood vessel in the gap portions 65 between stent segments 51 may become stretched/expanded in the major-axis dimension of the stent segments 51 in relatively low-pressure conditions, such that the blood vessel assumes a non-natural, at least partially non-circular shape in both the portions 63 thereof that overlap with the stent segments and in the gap portions 65 of the blood vessel, thereby introducing compliance return to the target blood vessel. The connecting arms 55 and/or the gaps longitudinally separating the stent segments 51 can allow for the stent implant to provide compliance-enhancing functionality without requiring full longitudinal coverage of the stent frames of the stent segments. Therefore, the implant 50 can serve to reshape the target blood vessel without requiring full circumferential and/or longitudinal coverage of the blood vessel.

The stent segments 51 may include internal or external coverings, such as fabric and/or polymer, fluid-tight covers. In some implementations, the coverings may be disposed on an outer diameter of the stent segment frames 51 in areas along the major-axis sides/walls of the stent segments 51 to distribute the contact load between the blood vessel 16 and the stent segment frames and/or to protect the blood vessel inner wall from abrasion or other damage from the stent segments due to the concentrated pressure/force at the major-axis ends/walls. While the stent segments 51 are illustrated as oval stents, it should be understood that any or all of the collection of modular stent segments 51 of the implant 50 may be circular stent segments.

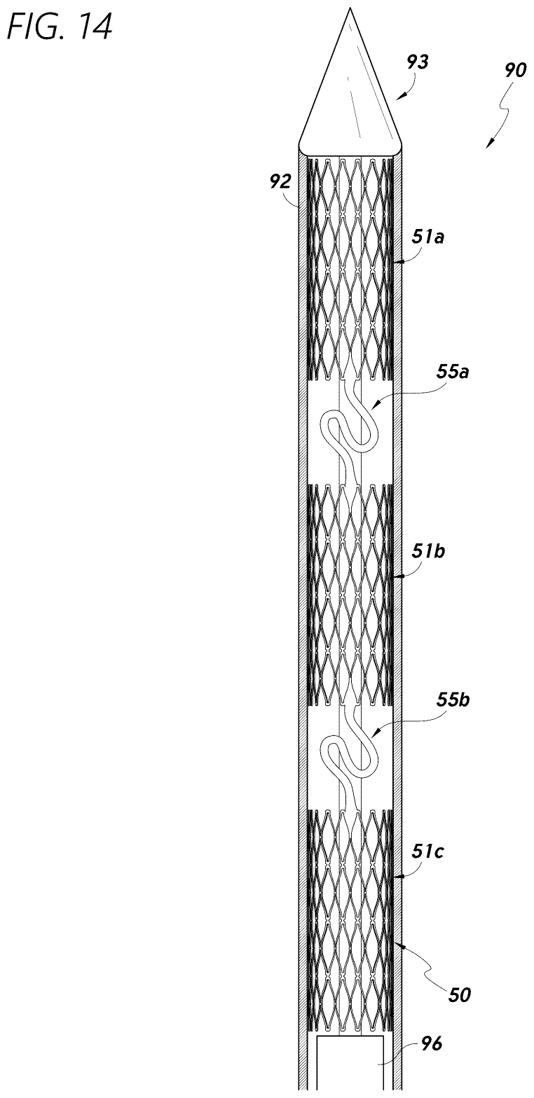

The modular stent implant 50 may be transported to the target implantation site using a catheter-based delivery system. An example delivery system 90 is shown in FIG. 14. The delivery system 90 may include an outer sheath 92, in which the implant device 50 may be disposed in a delivery configuration. For example, the stent segments 51 may be positioned within the outer sheath 92 in a series arrangement, as shown, wherein the connecting arms/struts 55 may effectively clocked the stent segments 51 in a common and/or desired orientation, which may facilitate deployment of the stent segments 51 in orientational alignment and/or in a desired orientation with respect to the target blood vessel.

The implant device 50 may be configured in a crimped/compressed delivery configuration within the delivery system 90, wherein the struts of the frames of the stent segments 51 may be generally more longitudinally arranged/aligned compared to expanded/deployed configurations thereof, thereby reducing the diametrical profile of the stent segments to fit within the outer sheath 92. In some implementations, the connecting arms/struts 55 may likewise be maintained in a compressed delivery configuration when disposed in the delivery system 90. For example, as shown in FIG. 14, the connecting arms/struts 55 may be longitudinally compressed, wherein one or more bends or curves in the struts/arms 55 may be implemented to reduce the length thereof. The shape memory of the arms/struts 55 may produce lengthening therein when the implant 50 is deployed from the delivery system 90, thereby increasing the overall length of the implant device 50.

In some implementations, the delivery system 90 comprises a nosecone feature 93, which may provide an atraumatic leading edge/component for the delivery system 90 to facilitate advancement through narrow and/or tortuous vasculature of the patient. The implant device 50 may be deployed by distally pushing a pusher device 96 relative to the outer sheath 92 and/or proximally pulling the outer sheath 92 relative to the pusher 96 to thereby pass the implant device 50 out of a distal opening/end of the outer sheath 92.

The delivery system may include certain features configured to align the orientation of the stent segments. For example, the delivery system may utilize a handle (not shown) or similar actuator/feature configured to be manipulated to align the major-axis plane of the implant 50 in a desirable position, such that the positioning of the stent implant 50 may be adjusted to the particular patient. Generally, the delivery system 90 may be utilized to implant the modular stent 50 in a manner such that the major-axis plane of the stent segments aligned more closely with the frontal/coronal plane than with the sagittal plane.

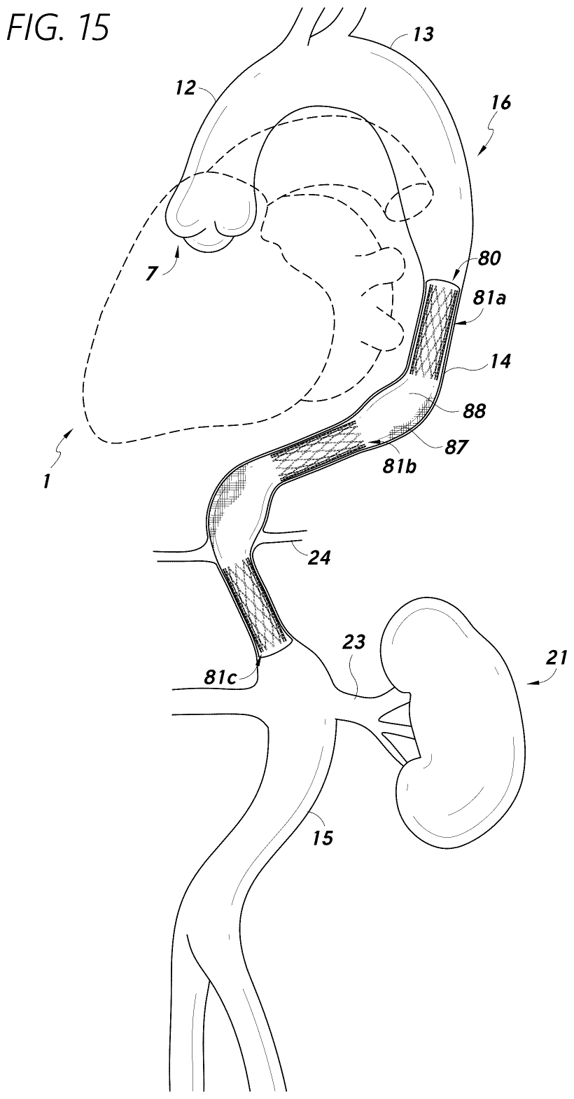

Although modular stent implants of the present disclosure are described in some examples as including stent segments coupled by connecting arm/struts that comprise as at least partially rigid structures, it should be understood that in some implementations, links/connections between adjacent stent segments may comprise portions of a covering. FIG. 15 shows a modular stent 80 implanted in a curved blood vessel segment in accordance with one or more examples, wherein the modular stent 80 comprises a plurality of stent segments 81 physically coupled to one another by portions 88 of a covering 87 that spans longitudinal/axial gaps between stent segments 81. For example, the modular stent 80 may comprise longitudinally-offset stent segments 81 physically coupled to one another by the covering 87, which may cover an inner and/or outer diameter/surface of one or more of the stent segments 81, wherein the covering 87 spans the distance between the stent segments. Such gap covering portions 88 may form a full tube of covering material, or one or more longitudinal bands, strips, or the like of covering material extending between a covering portion associated with one stent segment and a covering portion associated with a second stent segment. In some implementations, the covering 87 has one or more openings/cut-outs therein to allow for the passage of blood to side branch(es) through the implant 80.

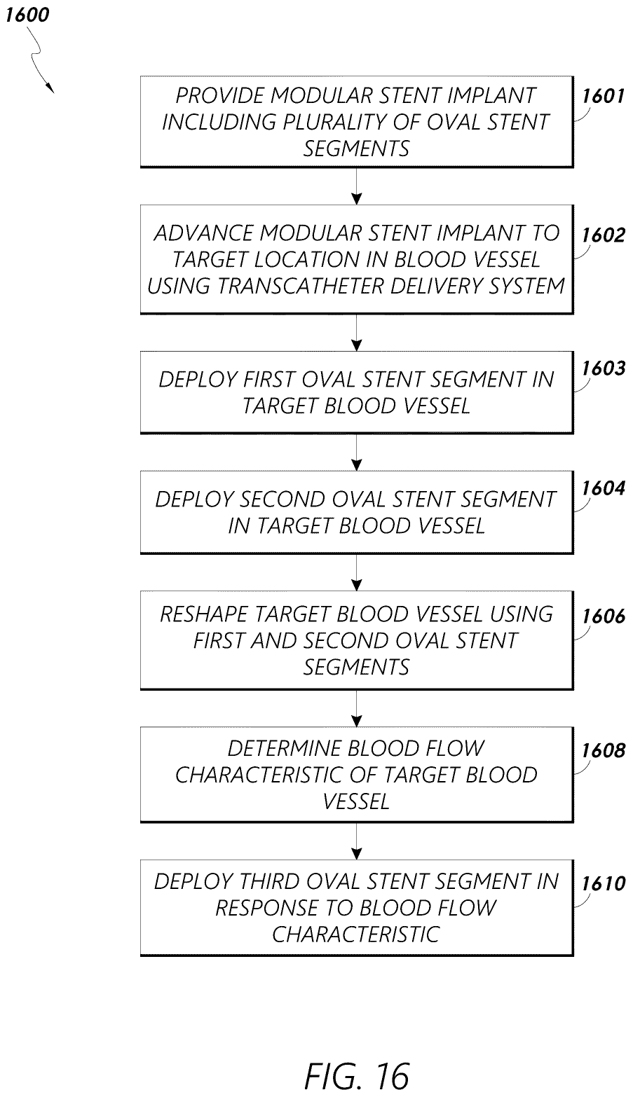

FIG. 16 is a flow diagram illustrating a process 1600 for managing blood flow in a target blood vessel of a patient. At block 1601, the process 1600 involves providing a modular stent comprising at least a first stent segment and a second stent segment, one or more of which may be a non-circular/oval stent segment. The first and second stent segments may be coupled by first and second coupling arms positioned on opposite circumferential portions of the first and second stent segments. In some implementations, the first and second stent segments are not coupled by connecting arms/struts.

At block 1602, the process involves advancing the modular stent implant to a target location in a blood vessel of a patient using a transcatheter delivery system to allow for deployment thereof at the target location in the blood vessel.

At block 1603, the process 1600 involves deploying/implanting the first oval stent segment in a target blood vessel, such as an ascending or descending portion of the aorta. At block 1604, the process involves deploying/implanting the second oval stent segment in the target blood vessel at an axially-offset position relative to the first oval stent segment. Deploying the modular stent can involve positioning the first oval stent segment and the second oval stent segment such that the first and second coupling arms span a curve in the blood vessel.

The modular stent may comprise only the first and second stent segments, or may comprise one or more additional stent segments, each of which may be coupled to one or more of the other stent segments of the modular stent by one or more coupling arms/struts as described herein. Deploying the first and second stent segments can involves bending coupling arm(s)/strut(s) between the first and second stent segments around a curve in the blood vessel. For example, bending the coupling arm(s)/strut(s) can involve bending hinge feature(s) of the arm(s)/strut(s). Where included/implemented, the coupling arm(s)/strut(s) of the modular stent can disposed on sides of the curve in the blood vessel when deployed/implanted. Deploying/implanting the first and second stent segments can involve positioning the first non-circular/oval stent segment and the second non-circular/oval stent segment such that at least one side branch blood vessel ostium is positioned/accessibly in a gap area between the first stent segment and the second stent segment.

At block 1606, the process 1600 involves cyclically reshaping the target blood vessel between more-circular and less-circular axial cross-sectional shapes using the implanted/deployed first oval stent segment and second oval stent segment, which may advantageously have shape memory characteristics configured to ovalize the target blood vessel when luminal pressure conditions permit.

At block 1608, the process 1600 involves determining a blood flow characteristic associated with the target blood vessel from said cyclical reshaping. The blood flow characteristic comprises a blood pressure waveform, which may be determined/generated using any suitable means/mechanism, such as a catheter-based sensor, a pressure cuff, or a sensor coupled to and/or otherwise associated with one or more of the deployed/implanted stent segments. The blood flow characteristic may comprise a flow and/or volume measurement with respect to the target blood vessel.

At block 1610, the process 1600 involves implanting a third oval stent segment in the target blood vessel at an axially-offset position relative to the second oval stent segment in response to the determined blood flow characteristic. For example, the third oval stent segment may be implanted such that a bend in the target blood vessel is present between the second oval stent segment and the third oval stent segment. The gap between the second stent segment and the third stent segment may accommodate the bend/curve in the blood vessel. The third oval stent segment can be implanted such that at least one side branch blood vessel ostium is positioned between the second stent segment and the third stent segment, such that the gap between the second and third stent segments provides a passage through which blood can supply the side branch(es).

As described in detail above, arterial stiffness can represent a relatively early detectable manifestation of adverse structural and/or functional changes within a blood vessel wall. While aortic stiffness can occur as a natural aging process, aortic stiffness can provide an independent predictor of coronary heart disease and/or stroke in relatively healthy subjects, and can further provide a predictor of mortality in the general population. Aortic stiffness is generally directly corelated with isolated systolic hypertension, a common form of systemic hypertension frequently encountered in elderly patients. Aortic stiffness can further be related to increased afterload in the left ventricle, potentially leading to left ventricular hypertrophy and/or remodeling. In some patients, aortic stiffness can ultimately lead, directly or indirectly, to the occurrence of heart failure.

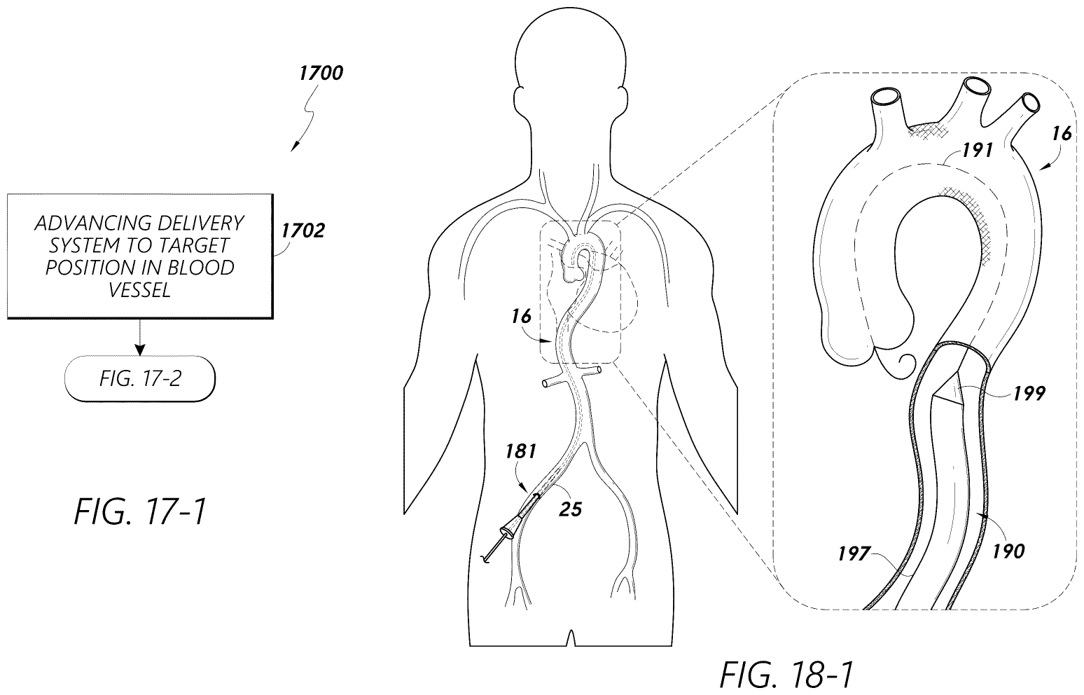

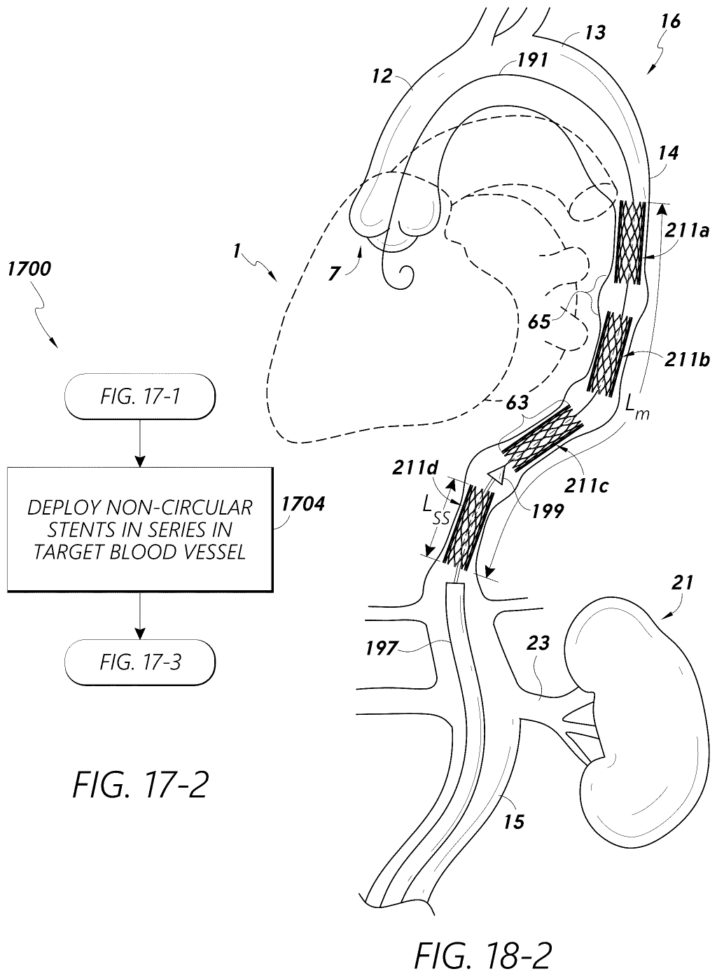

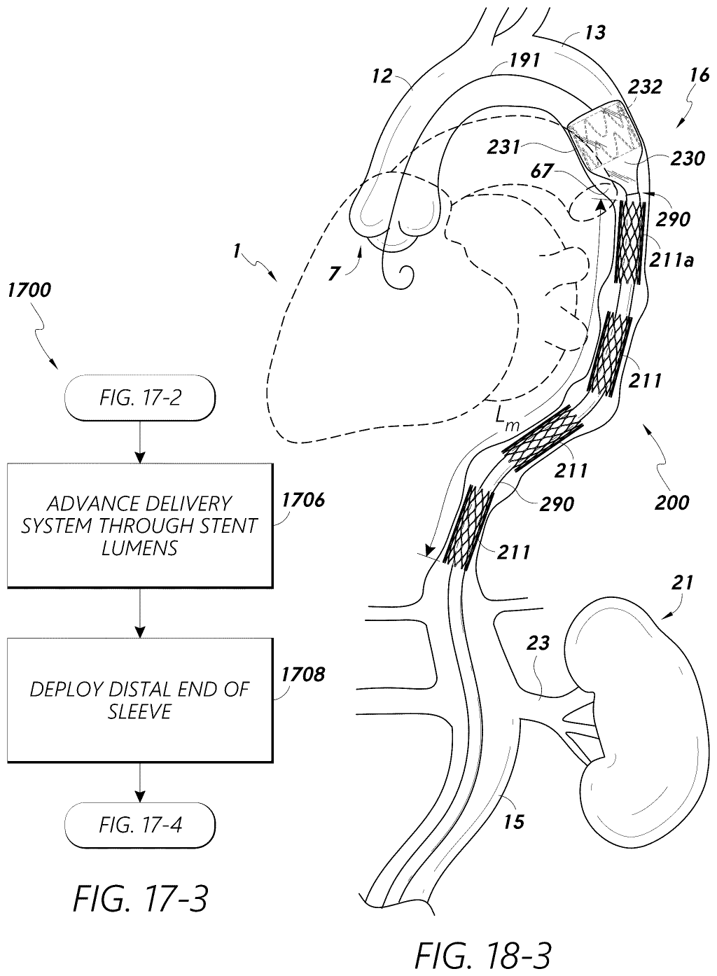

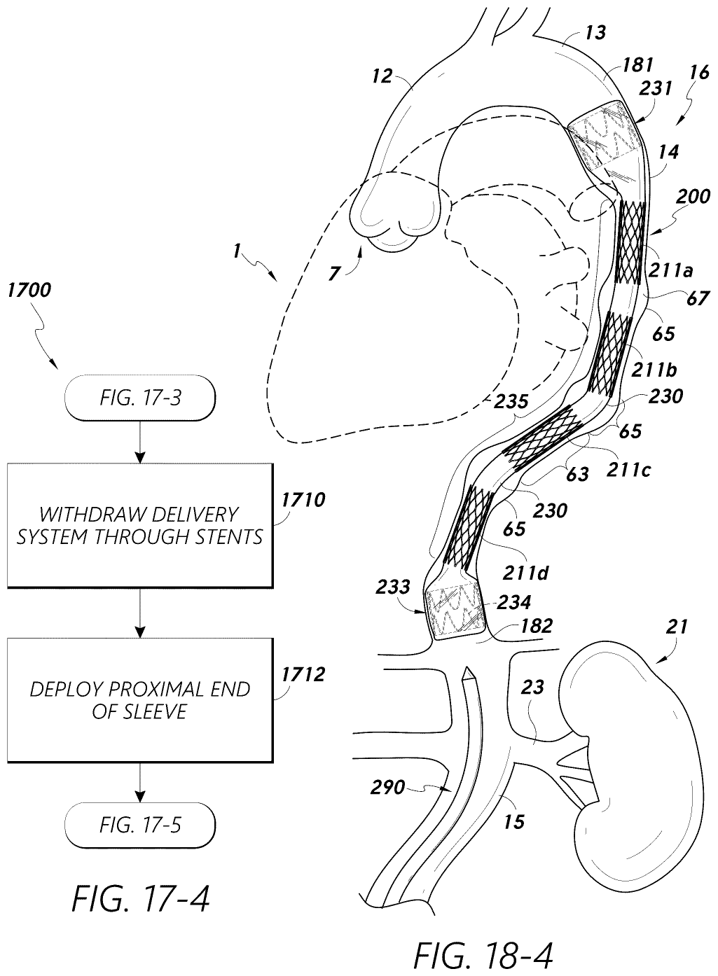

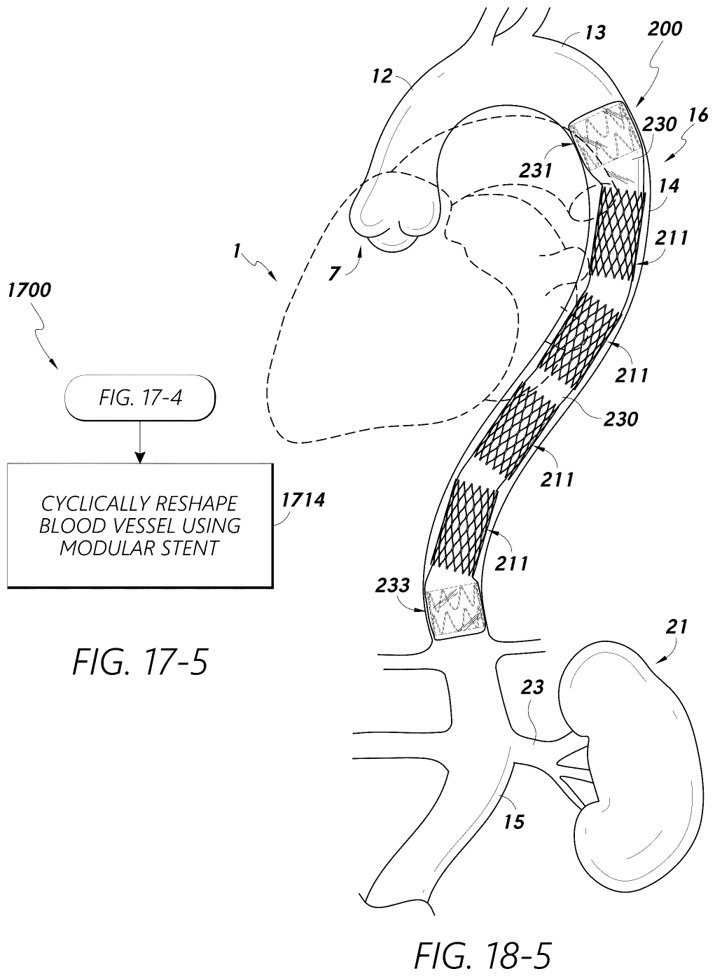

Restoring compliance to an otherwise stiff aorta using any of the example modular stent devices or processes disclosed herein can at least partially decrease/reduce left ventricular afterload, pulsatile pressure spikes, damage to the microvasculature of target organs (e.g., brain, kidneys), and/or provide other benefits. Various of the examples presented herein provide solutions for enhancing/increasing compliance in a target blood vessel, such as through the use of modular stents comprising oval/non-circular stent segments to alter the shape of the target blood vessel (e.g., aorta), wherein the geometrical shape change of the stent segments and aortic wall (e.g., from oval to circular) can be leveraged in order to restore compliance to the target blood vessel. However, with respect to such solutions, various difficulties can present relating to device sizing and/or necessary/desirable stent frame thickness. In order to provide a clinically-significant impact/improvement in blood flow characteristics (e.g., compliance), stent segments of a modular stent may need to be configured to promote a relatively significant volume shift between high-pressure (e.g., systole) and low-pressure (e.g., diastole) phases/conditions. Therefore, when implementing certain solutions, there may be a motivation to utilize relatively long and/or rigid stents/stent-segments, which, for reasons described in detail above, can present various technical challenges. In some implementations, examples of modular stents in accordance with aspects of the present disclosure include stent segments having inner tube/sleeve features, which can provide benefits relating to percutaneous deliverability, endovascular navigation, and/or other aspects. Aspects of modular stent implants comprising inner tube/sleeve features can be understood with respect to the process described in FIGS. 17-1-17-5.

FIGS. 17-1, 17-2, 17-3, 17-4, and 17-5 illustrate a flow diagram for a process 1700 for deploying a modular stent implant 200 including an inner sleeve 230 in accordance with one or more examples. FIGS. 18-1, 18-2, 18-3, 18-4, and 18-5 provide images of aspects of the modular stent 200, delivery system components, and anatomy relating to operations of the process 1700 of FIGS. 17-1, 17-2, 17-3, 17-4, and 17-5 according to one or more examples.

At block 1702, the process 1700 involves advancing a delivery system 190 to a target position in a blood vessel 16, such as the aorta. Endovascular access can be obtained via a transfemoral approach, as shown in FIG. 18-1. For example, the delivery system 190 may be advanced through a percutaneous introducer or other minimally-invasive access 181 into the vasculature of the patient, and further within the vasculature to a target position within the aorta 16 of the patient. The delivery system 190 may include one or more catheters/sheaths 197 and/or a nosecone 199 or other feature configured to facilitate atraumatic and/or smooth forward advancement of the delivery system 190 through tortuous anatomy of the vasculature. The percutaneous entry 181 may be at the femoral artery 25 or other arterial blood vessel, or through any other percutaneous transcatheter access path. The delivery system 190 may be advanced to the target implantation site over a previously-placed at least partially rigid guidewire 191.

A modular stent implant 200 (see, e.g., FIG. 18-4) comprising a plurality of stent segments 211 may be transported to the target implantation site in a delivery configuration in which the frames of the stent segments 211 are radially compressed. In some implementations, an elongate sleeve 230 can be configured to be transported in a delivery (e.g., bunched) configuration within the inner diameters/lumens of the stent segments 211, which may allow for simultaneous deployment of the stent segments 211 and sleeve 230 and/or may otherwise simplify delivery of such devices. Alternatively, the stent segments 211 and sleeve 230 may be delivered to the target anatomy at separate times, in separate delivery systems, and/or in axially offset positions within a common sheath or other delivery system component (e.g., catheter/sheath 197). In the example process 1700 described, the stent segments 211 and sleeve 230 are deployed separately.

At block 1704, the process 1700 involves deploying a plurality of non-circular stent segments 211 in a target blood vessel, such as the aorta 16. The non-circular/oval stents 211 can be deployed in the thoracic descending aorta 14 in some implementations. In the relevant figures, four stent segments 211a, 211b, 211c, 211d are shown. However, it should be understood that any number of stents/segments may be deployed/implanted in connection with the process 1700. The stent segments 211 may be deployed, individually, using a transcatheter process over the relatively stiff guidewire 191 for support and navigation. In some implementations, as described in detail herein, the stents/stent-segments 211 may be deployed in the target blood vessel 16 in a series arrangement with gaps 65 between longitudinally adjacent stents/segments (e.g., stent segments 211a, 211b; stent segments 211b, 211c; and stent segments 211c, 211d).

FIG. 18-2 shows four stent segments 211 deployed/implanted in the descending aorta 14, although other numbers of stents/segments may be implemented in connection with the process 1700 or any example disclosed herein. The stents 211 may be deployed using the catheter-based delivery system 190, which may be similar in one or more respects to any example delivery systems disclosed herein. The stent segments 211 may comprise relatively short stent frames comprising nitinol or other shape-memory material or at least partially rigid metal/material. The stent segments 211 may be covered or uncovered stents, as described herein. In some implementations, the stent segments 211 may have a length Lₛₛ of between 3-5 cm. The frames of the stent segments 211 can advantageously be shape-set to a non-circular/oval shape to provide a desired/optimized volume change between systole and diastole.

The separate stent segments 211 may be delivered independently, one-by-one, such as by using separate delivery systems, or re-using the same delivery system for each delivery/deployment. Alternatively, the stent segments 211 may be mounted together in/on the same delivery system 190 at a suitable axial offset distance between adjacent segments to accommodate bending and navigation of the delivery system 190 through tortuous anatomies. The delivery of any or all of the segments 211 can be performed using endovascular over-the-wire techniques.

Once deployed in the desired positions, with gap blood vessel segments 65 present between blood vessel segments 63 spanned by stent segments, the stents 211 may be configured to stretch the aorta/vessel sideways during low-pressure phases. For example, as deployed, the stent segments 211 may be configured to assume a non-circular (e.g., oval) shape in a relaxed, non-pressurized state thereof, thereby pushing the blood vessel walls radially-outwardly (e.g., at major-axis ends/walls of the stents) to reshape the vessel 16. As referenced above, the stent segments 211 may be transported to the implantation site in a radially-crimped/compressed configuration. The stent segments 211 may expand in accordance with self-expansion when deployed from the delivery system 190, or may be expanded using a balloon catheter or similar device. The stent segments 211 need not be excessively oversized relative to the blood vessel diameter. When placed in the blood vessel, the entire circumferences of the non-circular stent segments 211 may not be touching the inner diameter/circumference of the aortic wall, such that contact between the stent frames and the blood vessel wall may be primarily in the major-axis areas/walls of the stent segments with gaps in the areas of minor-axis areas/walls, as demonstrated in FIG. 7.

With the stents 211 in-place (e.g., in the thoracic descending aorta 14), an internal sleeve component 230 of the implant 200 may be deployed. At block 1706, the process 1700 involves advancing a delivery system 290 having the sleeve 230 disposed therein, wherein the delivery system 290 may or may not be the same delivery system 190 used to deploy the stent segments 211, through the internal lumens/channels of the deployed stents 211 and beyond a distal-most stent segment 211a, thereby traversing the blood vessel segment Lₘ spanned by the modular stents 211. After the stent segments 211 have been deployed, the relatively stiff guidewire 191 may still be maintained in position and traversing through the lumens of the stents 211. Therefore, the delivery system 290 may follow the guidewire 191 when moving into position to deploy the sleeve 230.

The sleeve 230 may advantageously be implanted in a manner as to achieve proximal and distal sealing of the sleeve 230. At block 1708, the process 1700 involves deploying a distal end/portion 231 of the sleeve component 230 of the modular stent 200 at and/or distal-to the end of the distal-most stent segment 211a of the modular stent. FIG. 18-3 shows the delivery system 290 positioned to deploy the distal portion 231 of the sleeve component 230 axially past the distal-most stent segment 211a. The distal portion 231 of the sleeve 230 may comprise a support frame 232 configured to provide a distal seal for the implant 200. The seal supports/portions of the sleeve 230 (e.g., the distal end portion 231) can provide a landing zone comprising one or more circumferential struts/rows to produce circumferential sealing of the end portion 231 against the inner diameter of the aortic wall to prevent blood from passing into the space/area 67 between the outer diameter of the sleeve 230 and the inner diameter of the vessel wall in the segment Lₘ.

To effect the seal at the distal (and/or proximal) end 231, the process 1700 may involve expanding a frame 232 or other sealing component associated with the distal end/portion 231 of the sleeve 230, wherein the frame/seal 232 prevents fluid passage radially outside of the sleeve 230 from the blood vessel distal of the implant 200. For example, the frame/seal 232 may comprise a wireframe stent form or similar structure configured to be expanded against the inner diameter of the blood vessel. Both the proximal and distal ends/aspects of the sleeve 230 may have sealing features, such as nitinol frames or other type of supports to provide circumferential apposition to the aortic wall, resulting in fluid-sealing.

At block 1710, the process 1700 involves proximally withdrawing the delivery system 290 back through the stents/segments 211 to a position proximal of the proximal-most stent/segment 211d. Withdrawing the delivery system 290 with the sleeve 230 anchored in place at the distal portion 231 can cause the medial portion 235 of the sleeve 230 to be deployed/drawn from the sheath/catheter of the delivery system 290 within the channels of the stent segments 211, wherein the sleeve 230 further spans the blood vessel gaps 65 between the stent segments 211.

At block 1712, the process 1700 involves deploying the proximal end/portion 233 of the sleeve component 230 at or proximal to the end of the proximal-most stent/segment 211d. The proximal end portion 233 may be similar to the distal end portion 231 and may include an expandable sealing frame 234 in some examples.

FIG. 18-4 shows the proximal portion/end of the sleeve 230 deployed, thereby completing implantation/deployment of the modular stent 200, including the various stent segments 211 and the sleeve 230 sealed against the inner diameter of the blood vessel wall and passing through the lumens of the serially-arranged stents 211. With the proximal end 233 sealed against the blood vessel inner diameter, the implant 200 provides a radially-sealed/closed fluid channel between the blood vessel segment 181 distal of the implant 200 and the blood vessel segment 182 proximal of the implant 200.

The medial portion/segment 235 of the sleeve 230 between the distal 231 and proximal 233 end portions may comprise the same material or different material compared to the end portions 231, 233. In some implementations, the medial portion 235 is flexible and/or elastic. For example, the sleeve 230 may be configured to radially expand and contract as luminal pressures change, thereby mimicking a compliant blood vessel. However, radial expansion of the medial portion 235 of the sleeve 230 can be limited by the inner diameters of the stents 211 and blood vessel walls 65, respectively.

At least a portion of the medial/middle portion 235, such as the entire medial portion 235, can be free of metal/rigid frame(s). For example, the medial portion 235 can comprise and/or consist of fabric or polymeric fluid-impermeable material. The absence of a frame in the medial portion 235 can allow for the sleeve 230 to assume a relatively low profile in a delivery configuration, which may allow for the use of a relatively low-profile delivery system 290. Except for the proximal 233 and distal 231 end sections, the remainder of the sleeve 230 can be flexible and unsupported and can be made of a fabric, tissue, and/or polymer.

At block 1710, the process 1700 involves cyclically reshaping the blood vessel using the implanted modular stent 200. Once the implant 200 is fully deployed, the circulatory blood flow can pass inside the sleeve 230, such that the sleeve 230 is subjected to the systemic pressure. The sleeve 230 can advantageously expand and engage circumferentially with the non-circular/oval stent segments 211, thereby allowing the stents 211 to cycle between less-circular and more-circular shapes, as described in detail herein. For example, the stents 211 can shift from oval to circular shapes according to the systolic and diastolic pressures, which can enhance/restore compliance to an otherwise stiff segment of the aorta or other target blood vessel.

With the medial portion 235 of the sleeve 230 disposed inside the stent segments 211, the arterial pressure in the blood vessel 16, which pressure level may likewise be present within the channel of the sleeve 230 due to fluid communication between the blood vessel 16 and the inner channel of the sleeve 230, may be transmitted directly to the sleeve 230 rather than the arterial wall in the area/segment covered/spanned by the implant 200. With the stent segments 211 disposed on the outside of the medial sleeve portion 235, the sleeve 235 can assume the biased, non-circular (e.g., oval) shape of the stent segments 211, to a greater degree in areas 63 overlapped by the stent segments 211 and possibly to a lesser degree in the gap areas 65, during diastole. As pressure is increased (e.g., during systole), the stent segments 211 and/or sleeve 230 may become more circularized, as shown in FIG. 18-5. In FIG. 18-5, the implant 200 is shown as implanted during a high-pressure phase of the cardiac cycle, wherein the luminal pressure in the sleeve 230 and target blood vessel 16 causes circularization of the stents 211 to increase the volume thereof for flow leveling in accordance with aspects of the present disclosure. During systole, the implant 200, due to the expansion thereof as illustrated in FIG. 18-5, may cause at least partial straightening in the aorta 16 or other target blood vessel, which may further improve blood flow.

### Additional Examples

Depending on the example, certain acts, events, or functions of any of the processes or algorithms described herein can be performed in a different sequence, may be added, merged, or left out altogether. Thus, in certain examples, not all described acts or events are necessary for the practice of the processes.

Provided below is a list of examples, each of which may include aspects of any of the other examples disclosed herein. Furthermore, aspects of any example described above may be implemented in any of the numbered examples provided below.

Example 1: A modular stent comprising a first oval stent segment, a second oval stent segment, and a first linking arm coupled to a first major-axis side of the first oval stent segment and to a first major-axis side of the second oval stent segment.

Example 2: The modular stent of any example herein, in particular example 1, wherein the first linking arm has a longitudinally-straight shape.

Example 3: The modular stent of any example herein, in particular example 1 or example 2, wherein the first linking arm has a longitudinally-curved shape.

Example 4: The modular stent of any example herein, in particular example 3, wherein the first linking arm has an ‘S’ shape.

Example 5: The modular stent of any example herein, in particular example 1, wherein the first linking arm has a zig-zag shape.

Example 6: The modular stent of any example herein, in particular any of examples 1-5, wherein the first linking arm comprises a hinge feature.

Example 7: The modular stent of any example herein, in particular example 6, wherein the first linking arm comprises a first strut associated with the first oval stent segment and a second strut associated with the second oval stent segment, and the first strut is coupled to the second strut by the hinge feature.

Example 8: The modular stent of any example herein, in particular any of examples 1-7, wherein the first linking arm has a greater radial dimension than circumferential dimension.

Example 9: The modular stent of any example herein, in particular any of examples 1-8, wherein the first linking arm is configured to bend in a dimension parallel with a minor axis of the first oval stent segment and the second oval stent segment.

Example 10: The modular stent of any example herein, in particular example 9, wherein the first linking arm is configured to resist deflection in a dimension parallel with a major axis of the first oval stent segment and the second oval stent segment.

Example 11: The modular stent of any example herein, in particular any of examples 1-10, further comprising a second linking arm coupled to a second major-axis side of the first oval stent segment and to a second major-axis side of the second oval stent segment.

Example 12: The modular stent of any example herein, in particular example 11, wherein the first linking arm and the second linking arm are configured to hold a gap portion of a blood vessel between the first oval stent segment and the second oval stent segment in a stretched shape with respect to a major-axis dimension of at least one of the first oval stent segment or the second oval stent segment.

Example 13: The modular stent of any example herein, in particular any of examples 1-12, further comprising a third oval stent segment coupled to the second oval stent segment by a second linking arm.

Example 14: The modular stent of any example herein, in particular example 13, wherein the second linking arm is coupled to the first major-axis side of the second oval stent segment and to a first major-axis side of the third oval stent segment.

Example 15: The modular stent of any example herein, in particular any of examples 1-14, wherein at least one of the first oval stent segment or the second oval stent segment comprises a covering.

Example 16: The modular stent of any example herein, in particular example 15, wherein the covering does not cover the first linking arm.

Example 17: The modular stent of any example herein, in particular any of examples 1-16, wherein the first linking arm is integrated with frames of the first oval stent segment and the second oval stent segment.

Example 18: The modular stent of any example herein, in particular example 17, wherein the first oval stent segment, the second oval stent segment, and the first linking arm are formed from a common metal tube.

Example 19: A method of managing blood flow, the method comprising implanting a first oval stent in a target blood vessel, implanting a second oval stent in the target blood vessel at an axially-offset position relative to the first oval stent, cyclically reshaping the target blood vessel between more-circular and less-circular axial cross-sectional shapes using the first oval stent and the second oval stent, determining a blood flow characteristic associated with the target blood vessel from said cyclical reshaping, and in response to the determined blood flow characteristic, implanting a third oval stent in the target blood vessel at an axially-offset position relative to the second oval stent.

Example 20: The method of any example herein, in particular example 19, wherein the third oval stent is implanted such that a bend in the target blood vessel is present between the second oval stent and the third oval stent.

Example 21: The method of any example herein, in particular example 19 or example 20, wherein the third oval stent is implanted such that at least one side branch blood vessel ostium is positioned between the second oval stent and the third oval stent.

Example 22: The method of any example herein, in particular any of examples 19-21, wherein the blood flow characteristic comprises a blood pressure waveform.

Example 23: A method of managing blood flow, the method comprising providing a modular stent comprising a first non-circular stent segment coupled to a second non-circular stent segment by first and second coupling arms positioned on opposite circumferential portions of the first non-circular stent segment and the second non-circular stent segment, advancing the modular stent to a target location in a blood vessel of a patient using a transcatheter delivery system, and deploying the modular stent at the target location in the blood vessel.

Example 24: The method of any example herein, in particular example 23, wherein said deploying the modular stent involves positioning the first non-circular stent segment and the second non-circular stent segment such that the first and second coupling arms span a curve in the blood vessel.

Example 25: The method of any example herein, in particular example 23 or example 24, wherein said deploying the modular stent involves bending the first and second coupling arms around a curve in the blood vessel.

Example 26: The method of any example herein, in particular example 25, wherein said bending the first and second coupling arms comprises bending hinge features of the first and second coupling arms, respectively.

Example 27: The method of any example herein, in particular example 25 or example 26, wherein, as deployed, the first and second coupling arms of the modular stent are disposed on sides of the curve in the blood vessel.

Example 28: The method of any example herein, in particular any of examples 23-27, wherein said deploying the modular stent involves positioning the first non-circular stent segment and the second non-circular stent segment such that at least one side branch blood vessel ostium is positioned between the first non-circular stent segment and the second non-circular stent segment.

Example 29: A modular stent implant device comprising first and second stents, each of the first and second stents having a non-circular axial cross-sectional shape in a relaxed state thereof, and first and second connecting arms coupled between the first and second stents and holding the first and second stents in rotational alignment.

Example 30: The modular stent implant device of any example herein, in particular example 29, wherein the first and second connecting arms emanate from major-axis vertices of the first and second stents, respectively.

Example 31: A modular stent assembly comprising a plurality of stents arranged in a series arrangement, and a sleeve extending through inner lumens of each of the plurality of stents.

Example 32: The modular stent assembly of any example herein, in particular example 31, further comprising a first seal associated with a first end of the sleeve, and a second seal associated with a second end of the sleeve.

Example 33: The modular stent assembly of any example herein, in particular example 32, wherein at least one of first seal or the second seal comprises an expandable frame.

Example 34: The modular stent assembly of any example herein, in particular example 32 or example 33, wherein the first seal and the second seal are positioned axially outside of the plurality of stents.

Example 35: The modular stent assembly of any example herein, in particular any of examples 32-34, wherein the sleeve comprises an elastic medial portion extending between the first seal and the second seal.

Example 36: The modular stent assembly of any example herein, in particular any of examples 31-35, wherein the sleeve comprises elastic material.

Example 37: The modular stent assembly of any example herein, in particular any of examples 31-36, wherein the sleeve is fluid-tight.

Example 38: The modular stent assembly of any example herein, in particular any of examples 31-37, wherein the sleeve comprises a fabric medial portion.

Example 39: The modular stent assembly of any example herein, in particular any of examples 31-38, wherein the sleeve comprises a polymer medial portion.

Example 40: The modular stent assembly of any example herein, in particular any of examples 31-39, wherein the sleeve comprises a medial portion formed of biological tissue.

Example 41: The modular stent assembly of any example herein, in particular example 31-40, wherein the plurality of stents comprises at least three stents.

Example 42: A method of managing blood flow, the method comprising accessing a target blood vessel via a transcatheter access, implanting a first oval stent in the target blood vessel, implanting a second oval stent in the target blood vessel at an axially-offset position relative to the first oval stent, and deploying a sleeve within a first lumen of the first oval stent and a second lumen of the second oval stent.

Example 43: The method of any example herein, in particular example 42, wherein the transcatheter access is through at least a portion of a femoral artery.

Example 44: The method of any example herein, in particular example 42 or example 43, wherein the target blood vessel comprises a descending thoracic portion of an aorta.

Example 45: The method of any example herein, in particular any of examples 42-44, further comprising, prior to said implanting the first oval stent, placing a guidewire in the target blood vessel, wherein said implanting the first oval stent, said implanting the second oval stent, and said deploying the sleeve are performed with the guidewire in-place in the target blood vessel.

Example 46: The method of any example herein, in particular any of examples 42-45, wherein said implanting the first oval stent and said deploying the sleeve are performed using a common delivery system.

Example 47: The method of any example herein, in particular any of examples 42-46, wherein said implanting the first oval stent and said deploying the sleeve are performed using separate delivery catheters.

Example 48: The method of any example herein, in particular any of examples 42-47, further comprising, prior to said deploying the sleeve, stretching the target blood vessel to a non-circular shape using at least one of the first oval stent or the second oval stent.

Example 49: The method of any example herein, in particular any of examples 42-48, wherein, prior to said deploying the sleeve, gaps are present between minor-axis walls of the first and second oval stents and an inner diameter of the target blood vessel.

Example 50: The method of any example herein, in particular any of examples 42-49, further comprising, prior to said deploying the sleeve, advancing a delivery system containing the sleeve through lumens of the first and second oval stents to a first position in the target blood vessel that is distal of the first oval stent.

Example 51: The method of any example herein, in particular example 50, further comprising deploying a distal portion of the sleeve at the first position in the target blood vessel, withdrawing the delivery system back through the lumens of the first and second oval stents to a second position in the target blood vessel that is proximal of the second oval stent, and deploying a proximal portion of the sleeve at the second position in the target blood vessel.

Example 52: The method of any example herein, in particular example 51, wherein the distal portion of the sleeve comprises an expandable frame.

Example 53: The method of any example herein, in particular example 52, further comprising expanding the expandable frame form a seal between the distal portion of the sleeve and the target blood vessel.

Example 54: The method of any example herein, in particular example 53, wherein the expandable frame has a circular axial-cross-sectional shape when expanded.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method of managing blood flow, the method comprising:
advancing a first delivery system to a segment of a descending aorta via an endovascular access path;
deploying a first stent from the first delivery system at a first position in the descending aorta, the first stent having a non-circular cross-sectional shape defining a major-axis diameter and a minor-axis diameter that is less than the major-axis diameter;
deploying a second stent from the first delivery system at a second position spaced from the first position by a first axial gap, the second stent having the non-circular cross-sectional shape and being physically coupled to the first stent by first and second coupling arms positioned on opposite major-axis circumferential portions of the first and second stents, respectively;
reducing systolic pressure in the descending aorta through circularization of the first and second stents, thereby storing energy in frames of the first and second stents; and
increasing diastolic pressure in the descending aorta through shape-memory return of the first and second stents to non-circular shapes;
wherein shape changes in the first and second stents assist with the management of blood flow.

2. The method of claim 1, further comprising deploying a third stent from the first delivery system at a third position spaced from the second position by a second axial gap, the third stent having the non-circular cross-sectional shape and being physically coupled to the second stent by third and fourth coupling arms positioned on the opposite major-axis circumferential portions of the second stent, respectively.

3. The method of claim 2, wherein the first, second, and third stents span multiple bends in the descending aorta.

4. The method of claim 1, further comprising bending the first and second coupling arms around a curve in the descending aorta.

5. The method of claim 1, wherein deploying the first stent and deploying the second stent involve positioning the first stent and the second stent within the descending aorta such that at least one side branch blood vessel ostium is positioned in the first axial gap between the first stent and the second stent.

6. The method of claim 1, wherein the first and second coupling arms provide the only physical coupling between the first stent and the second stent.

7. The method of claim 1, further comprising, after deploying the first stent and the second stent:
advancing a second delivery system through interior lumens of the second stent and the first stent;
deploying a first support frame from the second delivery system;
anchoring the first support frame to an inner diameter of a first blood vessel segment distal of the first stent, the first support frame being coupled to a tubular sleeve; and
withdrawing the second delivery system back through the interior lumens of the first stent and the second stent, thereby deploying the tubular sleeve within the interior lumens of the first stent and the second stent.

8. The method of claim 7, further comprising:
deploying a second support frame from the second delivery system; and
anchoring the second support frame to an inner diameter of a second blood vessel segment proximal of the second stent, the second support frame being coupled to the tubular sleeve, thereby providing a sealed fluid channel between the first support frame and the second support frame and through the interior lumens of the first stent and the second stent.

9. The method of claim 8, wherein the first support frame and the second support frame have circular cross-sectional shapes.

10. The method of claim 7, wherein the first delivery system and the second delivery system are a common delivery system.

11. The method of claim 1, wherein the first stent and the second stent have peanut-shaped axial cross-sections with a first minor-axis diameter at opposite outer portions along a major-axis of the respective first and second stents and a second minor-axis diameter at a central position between the opposite outer portions, the second minor-axis diameter being less than the first minor-axis diameter.

12. A method of managing blood flow, the method comprising:
providing a modular stent comprising a first non-circular stent coupled to a second non-circular stent by first and second coupling arms positioned on opposite circumferential portions of the first non-circular stent and the second non-circular stent;
advancing the modular stent to a target location in a blood vessel of a patient using a transcatheter delivery system;
deploying the modular stent at the target location in the blood vessel; and
cyclically reshaping the blood vessel using the modular stent to improve blood circulation in the patient.

13. The method of claim 12, wherein deploying the modular stent involves bending the first and second coupling arms around a curve in the blood vessel.

14. The method of claim 12, wherein deploying the modular stent involves positioning the first non-circular stent and the second non-circular stent such that at least one side branch blood vessel ostium is positioned between the first non-circular stent and the second non-circular stent.

15. The method of claim 12, further comprising deploying a sleeve within a first lumen of the first non-circular stent and a second lumen of the second non-circular stent.

16. A method of improving blood flow in a patient, the method comprising:
providing a modular stent assembly including:
a first self-expanding oval stent including struts forming a first plurality of rows of cells;
a second self-expanding oval stent including struts forming a second plurality of rows of cells; and
a first linking arm coupled to a first major-axis side of the first oval stent and to a first major-axis side of the second oval stent;
deploying the first self-expanding oval stent and the second self-expanding oval stent in a blood vessel, wherein the first and second self-expanding oval stents become more circular under increased blood pressure and wherein a more circular shape increases a volume of blood flow through the first and second self-expanding oval stents; and
cyclically reshaping the blood vessel between more-circularized and less-circularized shapes using the first self-expanding oval stent and the second self-expanding oval stent, thereby moderating pressure waves within the blood vessel across multiple cardiac cycles.

17. The method of claim 16, wherein the first linking arm has a longitudinally straight shape.

18. The method of claim 16, wherein the first linking arm has an ‘S’ shape or a zig-zag shape.

19. The method of claim 16, wherein:
the first linking arm comprises a first strut associated with the first oval stent and a second strut associated with the second oval stent; and
the first strut is coupled to the second strut by a hinge feature.

20. The method of claim 16, wherein:
the first linking arm is configured to bend in a dimension parallel with a minor axis of the first oval stent and the second oval stent; and
the first linking arm is configured to resist deflection in a dimension parallel with a major axis of the first oval stent and the second oval stent.
